(19) United States
(12) Patent Application Publication
(10) Pub. No.: US 2008/0305085 A1
(43) Pub. Date: Dec. 11, 2008

(54) Compositions And Methods For Stem Cell Expansion

(76) Inventors: David T. Scadden, Weston, MA (US); Sebastian Stier, Bonn (DE)

Correspondence Address: EWARDS ANGELL PALMER & DODGE LLP, P.O. BOX 55874, BOSTON, MA, 02205, US

(21) Appl. No.: 11/791,147

(22) PCT Filed: Nov. 18, 2005
(86) PCT No.: PCT/US05/41927

§ 371 Date: Aug. 20, 2008

Related U.S. Application Data
(60) Provisional application No. 60/629,626, filed on Nov. 19, 2004.

(51) Int. Cl. A61K 35/12; C12N 5/06; A61K 38/00; A61P 31/00

(52) U.S. Cl. 424/93.7; 435/374; 514/12

(57) ABSTRACT

The present invention features methods and compositions that are useful for promoting stem cell survival and expansion. In addition, the invention also provides compositions and methods for the treatment of neoplasia.

### CROSS-REFERENCE TO RELATED APPLICATION

This application claims the benefit of U.S. provisional application 60/629,626, filed on Nov. 19, 2004, which is hereby incorporated by reference in its entirety. Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

This work was supported by a National Institutes of Health Grant No. RO1 HL44851. The government may have certain rights to the invention.

### BACKGROUND OF THE INVENTION

Stem cell fate is influenced by specialized microenvironments, or niches. The stem cell niche is a specialized microenvironment that houses and regulates the stem cell pool. In lower organisms, the niche incorporates elements that support a primitive or stem cell phenotype and distinct anatomic components that enforce terminal differentiation and end cell cycling among stem cell progeny. In this way, the Drosophila melanogaster germ cell niche both nurtures and constrains stem cells, maintaining strict control on stem cell number. Whether the same is true for mammalian stem cell niches has not been well defined. As cellular members of mammalian stem cell niches are characterized, strategies for modulating the niche to achieve therapeutic outcomes becomes feasible. Niche constituent cells or signalling pathways provide pharmacological targets with therapeutic potential for stem-cell-based therapies.

### SUMMARY OF TEE INVENTION

As described below, the present invention features methods and compositions that are useful for promoting stem cell survival and expansion or for treating a neoplasia.

In one aspect, the invention generally features methods of promoting stem cell survival or generation. The method involves contacting a stem cell or stem cell progenitor, and a support cell that expresses osteopontin (OPN) with an OPN inhibitor; and growing the stem cell or stem cell progenitor in the presence of the support cell, where the method promotes stem cell survival or generation. In one embodiment, the stem cell is selected from the group consisting of a mesenchymal, skin, neural, intestinal, liver, cardiac, prostate, mammary, kidney, pancreatic, retinal and lung stem cell. In another embodiment, the stem cell is a hematopoietic stem cell. In yet another embodiment, the support cell is a cellular component of a stem cell niche. In yet another embodiment, the support cell is an osteoblast.

In another embodiment, the generation is by stem cell self-renewal. In yet another embodiment, the generation is by proliferation or differentiation of the stem cell progenitor. In yet another embodiment, the method reduces apoptosis. In yet another embodiment, the method is carried out in vivo or in vitro.

In another aspect, the invention features a method of promoting stem cell survival or generation. The method involves contacting a stem cell or stem cell progenitor that expresses osteopontin (OPN) with an OPN inhibitor; and growing the stem cell or stem cell progenitor, where the method promotes stem cell survival or generation.

In yet another aspect, the invention features a method of promoting hematopoietic stem cell survival or generation. The method involves contacting a hematopoietic stem cell or hematopoietic stem cell progenitor, and a support cell that expresses osteopontin (OPN) with an OPN inhibitor; and growing the hematopoietic stem cell or hematopoietic stem cell progenitor in the presence of the support cell, where the method promotes hematopoietic stem survival or generation.

In another aspect, the invention provides a method of increasing the number of self-renewing stem cells in a subject in need thereof. The method involves the steps of contacting an isolated population of cells that comprises at least stem cells and support cells with an OPN inhibitor; and administering the cells to the subject, thereby increasing the amount of self-renewing stem cells in the subject. the cells are obtained from the subject. In one embodiment, the subject is a human. In another embodiment, the cells are administered to the subject during a bone marrow transplant. In yet another embodiment, the cells are obtained from bone marrow. In yet another embodiment, the bone marrow cells comprise an osteoblast, a hematopoietic stem cell. In another embodiment, the bone marrow cells comprise a Lin⁻ cKit⁺Sca1⁺. In yet another embodiment, the method further includes contacting the stem cell or support cell with parathyroid hormone.

In another aspect, the invention features a method for enhancing engraftment of a stem cell into a tissue of a subject. The method involves contacting a tissue of a subject with an OPN inhibitor; and providing a stem cell to the tissue, thereby enhancing engraftment of the stem cell into the tissue of the subject.

In yet another aspect, the invention features a method of modulating a stem cell niche, the method involving contacting the niche with an OPN inhibitor, thereby modulating the stem cell niche. In one embodiment, the stem cell niche comprises at least one cell that expresses OPN (e.g., a bone marrow stromal cell). In other embodiments, the stem cell niche comprises any one or more of a fibroblast, an osteoblast, an adipocyte, an endothelial cell, and a macrophage.

In another aspect, the invention features a method for enhancing the hematopoietic stem cell-proliferating activity of a stromal cell. The method involves contacting the stromal cell with an OPN inhibitor. In one embodiment, the stromal cell is an osteoblast. In other embodiments, the stromal cell is contacted in vivo or in vitro.

In another aspect, the invention features a method for enhancing engraftment of a hematopoietic stem cell into the bone marrow of a subject. The method involves contacting an isolated bone marrow derived cell with an OPN inhibitor; and providing the bone marrow derived cell to a subject, thereby enhancing engraftment of the stem cell into the tissue of the subject.

In another aspect, the invention features a method of enhancing engraftment of a hematopoietic stem cell into bone marrow of a subject. The method involves providing a stem cell or stem cell progenitor and a bone marrow-derived cell expressing an OPN inhibitory nucleic acid molecule to a subject, where the method enhances engraftment of the stem cell into the bone marrow of the subject.

In another aspect, the invention features a method of identifying a candidate compound that promotes stem cell survival or generation. The method involves contacting a cell that expresses OPN with a candidate compound; and detecting a decrease in OPN expression or activity, where the decrease identifies a candidate compound that promotes stem cell survival, differentiation, or proliferation. In one embodiment, the method further includes the step of identifying an increase in stem cell number. In another embodiment, the candidate compound reduces the expression of OPN. In another embodiment, the candidate compound reduces the biological activity of OPN. In yet another embodiment, the cell is obtained from a subject and is a bone marrow cell (e.g., an osteoblast).

In another aspect, the invention features an expression vector comprising a promoter operably linked to a nucleic acid encoding an OPN inhibitory nucleic acid molecule, where the promoter is sufficient to direct expression of the OPN inhibitory nucleic acid molecule in a bone marrow derived cell. In one embodiment, the promoter is an osteoblast specific collagenα1(I) promoter. In another embodiment, the inhibitory nucleic acid molecule is an siRNA, shRNA, or anti-sense RNA.

In another aspect, the invention features isolated bone marrow derived cell containing an OPN inhibitory nucleic acid molecule, where the OPN inhibitory nucleic acid molecule reduces expression of OPN in the cell. In one embodiment, the cell is a stromal cell. In another embodiment, the cell is an osteoblast.

In another aspect, the invention features kit for promoting stem cell survival, growth, or proliferation containing an OPN inhibitor, and instructions for using the inhibitor to promote stem cell survival, growth, or proliferation.

In another aspect, the invention features a kit for enhancing engraftment of a stem cell into a tissue of a subject containing a cell that expresses OPN, containing an OPN inhibitor, and instructions for using the inhibitor to enhance engraftment of a stem cell into a tissue of a subject.

In various embodiments of any of the above aspects, the support cell is derived from bone marrow or is an osteoblast. In yet other embodiments of any of the above aspects, the stem cell generation is by hematopoietic stem cell self-renewal or by proliferation or differentiation of a hematopoietic stem cell progenitor. In yet other embodiments, the OPN inhibitor reduces OPN transcription, OPN translation, or reduces OPN biological activity. In yet other embodiments of the above aspects, the OPN inhibitor increases expression of angiopoietin-1 or Jag-1 or reduces apoptosis. In yet other embodiments of the above aspects, the OPN inhibitor is a small molecule, polypeptide (e.g., an antibody that specifically blocks an OPN interaction with an OPN receptor or an antibody that specifically binds an OPN polypeptide), or nucleic acid molecule (e.g., an siRNA, shRNA, or antisense RNA molecule). In yet other embodiments of the above aspects, the stem cell, stem cell progenitor or support cell is contacted ex vivo or in vivo. In yet other embodiments, the stem cell, stem cell progenitor or support cell in contacted with a parathyroid hormone. In yet other embodiments of the above aspects, the stem cell is selected from the group consisting of a mesenchymal, skin, neural, intestinal, liver, cardiac, prostate, mammary, kidney, pancreatic, retinal and lung stem cell. In yet other embodiments of any of the above aspects, the OPN inhibitor increases the ability of the niche to support stem cell survival, self-renewal, or generation. In yet other embodiments of any of the above aspects, the stem cell is a mesenchymal, skin, neural, intestinal, liver, cardiac, prostate, mammary, kidney, pancreatic, retinal and lung stem cell.

In another aspect, the invention features method of inhibiting the survival or proliferation of a neoplastic cell, the method involving contacting a neoplastic cell with an effective amount of an OPN polypeptide or analog thereof, where the method inhibits the survival or proliferation of the neoplastic cell.

In another aspect, the invention features method of inducing apoptosis in a neoplastic cell, the method involving contacting a neoplastic cell with an effective amount of an OPN polypeptide or analog thereof, where the method induces apoptosis in the neoplastic cell.

In various embodiments of the above aspects, the neoplastic cell is ill vivo or in vitro. In yet other embodiments, the neoplastic cell is in a subject diagnosed as having a neoplasia selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, myelodysplastic syndrome, and chronic lymphocytic leukemia.

In another aspect, the invention features a method of treating or preventing a neoplasia in a subject in need thereof, the method involving contacting a cell of the subject with a pharmaceutical composition involving an effective amount of an OPN polypeptide or analog thereof, where the method treats or prevents a neoplasia. In one embodiment, the subject is diagnosed as having a neoplasia selected from the group consisting of acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, myelodysplastic syndrome, and chronic lymphocytic leukemia.

In yet another aspect, the invention features a method of treating or preventing a neoplasia in a subject in need thereof, the method involving contacting a cell of the subject with a pharmaceutical composition involving an effective amount of a compound that increases the expression of an OPN polypeptide or nucleic acid molecule, where the method treats or prevents a neoplasia in the subject.

In yet another aspect, the invention features a method for identifying a compound that inhibits the survival or proliferation of a neoplastic cell. The method involves contacting a cell expressing an OPN nucleic acid molecule with a candidate compound; and measuring an increase in expression of the OPN nucleic acid molecule relative to a reference, where an increase in expression of the OPN nucleic acid molecule inhibits the survival or proliferation of a neoplastic cell.

In yet another aspect, the invention features a method for identifying a compound that inhibits the survival or proliferation of a neoplastic cell, the method involving contacting a cell expressing an OPN polypeptide with a candidate compound; and measuring an increase in expression of the OPN polypeptide relative to a reference, where an increase in expression of the OPN polypeptide inhibits the survival or proliferation of a neoplastic cell.

In embodiments of the above aspects, the compound increases OPN transcription or translation.

In another aspect, the invention features a method for identifying a compound that inhibits the survival or proliferation of a neoplastic cell, the method involving contacting a cell expressing an OPN polypeptide with a candidate compound; and measuring an increase in the biological activity of the OPN polypeptide relative to a reference following contact with the candidate compound, where an increase in expression of the OPN nucleic acid molecule inhibits the survival or proliferation of a neoplastic cell. In one embodiment, biological activity is measured in an immunoassay or enzymatic assay.

In another aspect, the invention features a method for diagnosing a patient as having, or having a propensity to develop, a neoplasia, the method involving determining an increased level of expression of an OPN nucleic acid molecule or polypeptide in a patient sample, where an increased level of expression relative to a reference, indicates that the patient has or has a propensity to develop a neoplasia.

In another aspect, the invention features an expression vector containing a promoter operably linked to a nucleic acid encoding an OPN nucleic acid molecule, where the promoter is sufficient to direct expression of the OPN nucleic acid molecule in a neoplastic cell.

In another aspect, the invention features a kit for inhibiting the survival, growth, or proliferation of a neoplastic cell, the kit containing an OPN polypeptide or nucleic acid molecule, and instructions for using the polypeptide or nucleic acid molecule to inhibit the survival, growth, or proliferation of a neoplastic cell.

In one aspect, the invention provides methods and compositions for expanding a stem cell population. In another aspect, the invention features methods and compositions for treating a neoplasia. Other features and advantages of the invention will be apparent from the detailed description, and from the claims.

### BRIEF DESCRIPTION OF THE DRAWINGS

FIGS. 1A and 1B show that bone marrow osteopontin production is altered by parathyroid hormone receptor (PTHr) activation on osteoblasts. FIG. 1A is a set of four micrographs showing that OPN is increased in bone marrow following activation of osteoblasts. Immunohistochemistry of tibia sections from wild-type (left) or littermate transgenic mice (right) with a constitutively activated parathyroid hormone/parathyroid related peptide receptor driven by a 2.3 kb fragment of the collagenα1(1) promoter. Sections were stained with antibody to osteopontin (red) and counterstain as described and photographed at 200× magnification (top panels) with ˜4× image blow-up in lower panels. Arrows indicate OPN rich spindle shaped cells lining the trabecular bone consistent with an osteoblast morphology. FIG. 1B shows reverse transcription-polymerase chain reaction (RT PCR) products separated on an agarose gel. This was used to analyse changes in OPN expression in Lin-ckit⁺Sca-1⁺ (LKS) cells treated with IL-Mix, SCF, IL3, IL6, G-CSF and GM-CSF at specified time points, as compared with the stable expression of GAPDH at those same time points. “6 h Control” denotes a control condition, where the cells were not treated with IL-Mix. “−RT” denotes a control condition without addition of reverse transcriptase to the reaction.

FIGS. 2A-2G are graphs showing that the primitive cell pool increased in OPN deficient mice. FIG. 2A shows the results of an analysis of bone marrow (BM) of OPN^{-/-} mice and littermate controls. The bone marrow cells were harvested, counted and stained with lineage specific markers CD4, CD8, B220, Mac1 (CD11b), Gr-1 and Ter119 prior to flow cytometry. The graph shows the average percentage±SEM (n=3). FIG. 2B is a dot plot that shows the results of an analysis of bone marrow cells of OPN^{+/+} and OPN^{-/-} mice that were stained with Sca1, c-kit and lineage markers (CD3, CD4, CD8, B220, Gr-1, CD11b and Ter119) for flow cytometry. The dot plots show the Sca1⁺ c-kit⁺ cells in the upper right quadrant gated on lin⁻ bone marrow cells for a single experiment. FIG. 2C is a graph showing that there is no significant change in the relative levels of IgM⁻ and IgM⁺ B220⁺ cells in either OPN^{+/+} or OPN^{-/-} mice. FIG. 2D is a graph that provides a summary of results for six mice in each group analysed as in FIG. 2B. FIG. 2E is a graph showing the absolute number cell numbers of highly stem cell enriched CD34-portion of the Sca1⁺ c-kit⁺lin⁻ in 8 pairs of control and littermate OPN^{-/-} mice as analysed by flow cytometry. FIG. 2F shows the results of long-term culture initiating cell assays. To confirm the immuno-phenotypic findings long-term culture initiating cell (LTC-IC) assays were performed at limiting dilution and the frequency of LTC-IC was calculated; data shown are the average frequency±SEM of LTC-ICs per 100,000 bone marrow cells (P=0.01, n=5 pairs). FIG. 2G shows the results of flow cytometry analyzing the contribution of Ly5.2 and Ly5.1 cells to the bone marrow of recipient mice. To confirm the LTC-IC data equal numbers of OPN deficient bone marrow (Ly5.2) and wild-type bone marrow of congenic mice (Ly5.1) were transplanted into lethally irradiated wild-type recipients in a competitive repopulation assay (CRA). Twelve weeks after transplantation the bone marrow of the recipient mice were analyzed for the contribution of Ly5.2 and Ly5.1 cells by flow cytometry with results shown (n=8).

FIGS. 3A-3E are graphs showing that OPN^{-/-} hematopoietic stem cell increase is not cell autonomous, but stroma dependent. FIG. 3A shows the results of a serial transplantation experiment using C57BL/6 wild-type mice (Ly5.1) as recipients for either OPN^{-/-} or OPN^{+/+} bone marrow (Ly5.2). “BMT” denotes bone marrow transplant. This experiment indicated that OPN^{-/-} hematopoietic stem cells lost their advantage in numbers by the second transplantation, reverting to the OPN^{+/+} phenotype. Data are presented as the ratio of OPN^{-/-} to OPN^{+/+} mean absolute number Sca1⁺c-kit⁺lin⁻ cells from 5 mice in each genotype at each transplantation. FIG. 3B shows that OPN^{-/-} primitive hematopoietic cells have no advantage in homing to the bone marrow. Whole bone marrow cells of male OPN^{-/-} and OPN^{+/+} mice (Ly5.2) were transplanted into lethally irradiated female recipients (Ly5.1) and sixteen hours after transplantation the bone marrow of the recipients was analyzed for Ly5.1 and Ly5.2 and differentiation markers. The chart shows that the approximately two-fold increase in donor OPN^{-/-} Sca1⁺c-kit⁺lin⁻ cells was preserved in the marrow of recipients. FIGS. 3C, 3D, and 3E show that primitive cell expansion in OPN^{-/-} mice is stroma-dependent. In FIG. 3C Sca1⁺lin⁻ hematopoietic stem cells were isolated from wild-type bone marrow and plated on either wild type or OPN deficient stroma in limiting dilution LTC-IC assays as described. (n=7). In FIGS. 3D and 3E wild-type bone marrow was transplanted into lethally irradiated OPN^{+/+} or OPN^{-/-} recipients. “Rec” denotes recipient. Twelve weeks after transplantation the bone marrow of the recipient mice were analyzed by flow cytrometric analyses and functional LTC-IC assays (n=4 for each assay).

FIGS. 4A-4F are graphs showing that OPN^{-/-} bone marrow has unaltered cell cycle profiles associated with increased stromal Jagged1 and Angiopoietin-1 expression and reduced primitive cell apoptosis. FIG. 4A is a graph showing that bone marrow Sca1⁺ c-kit⁺lin⁻ cells that show bright staining for Hoechst33342 are cells in the G2/M phase of the cell cycle (n=3 pairs). FIG. 4B shows BrdU incorporation in Sca1⁺c-kit⁺lin⁻ (KLS) cells at the specified time points in OPN^{+/+} and OPN^{-/-} bone marrow. Data are the result of two independent experiments with four mice per group in each experiment. Student t test comparison revealed no P<0.05. FIG. 4C shows that bone marrow adherent stromal cells were evaluated for Jagged1, Angiopoietin-1 (Ang-1) and N-cadherin expression by RT-PCR (n=6 for each). Data were normalized to an intrasample GAPDH standard and the results of the OPN^{-/-} versus OPN^{+/+} cells were compared by ratio. FIG. 4D shows Jagged 1 expression in wild-type bone marrow stroma treated with or without OPN 1 ug/ml for four hours and measured by RT PCR. Data are normalized against GAPDH expression measured by RT PCR. FIG. 4E shows that bone marrow cells of OPN and OPN mice were stained with antibodies to differentiation markers, the apoptosis marker AnnexinV and the DNA-dye 7-AAD. AnnexinV-positive/7-AAD-negative apoptotic Sca1⁺c-kit⁺lin⁻ cells are shown (n=4 pairs). FIG. 4F shows that the stroma dependent apoptotic rate demonstrated by reduced apoptosis of wild type primitive hematopoietic cells when transplanted into OPN−/− mice compared with OPN^{+/+} recipient mice. Analyses were performed on the lin⁻ fraction twelve weeks after transplantation (n=4).

FIGS. 5A-5C are graphs showing that soluble OPN induces apoptosis of primitive hematopoietic cells. Sca1⁺lin⁻ cells were isolated from the bone marrow of C57B1/6 mice and cultured in IMDM containing 10% fetal calf serum (FCS), stem cell factor (SCF), Flt-3, thrombopoietin (TPO) and IL-3 with or without OPN [1 μg/ml]. After 7 days the cells were counted and analyzed in functional hematopoietic assays. FIG. 5A shows that soluble OPN did not alter the absolute number of colony-forming cells (CFCs) per well in comparison to controls. Chart shows the total number of CFCs per well of 5 independent experiments (solid lines) and the mean of all experiments (dotted line). FIG. 5B shows that decreased primitive cell activity is detected in cells stimulated with OPN in comparison to controls. Chart shows the total number of LTC-ICs per well of 5 independent experiments (solid lines) and the mean of all experiments (dotted line). FIG. 5C shows that cultured cells were stained with lineage markers, AnnexinV and the DNA dye 7-AAD. The chart shows the average percentage±SEM of lin⁻7-AAD⁻AnnexinV⁺ cells representing apoptotic primitive hematopoietic cells.

FIG. 6 is a graph showing that OPN deficiency permits increased primitive hematopoietic cell compartment expansion after niche activation by parathyroid hormone (PTH): OPN^{+/+} and OPN^{-/-} mice were treated with PTH by daily injection for 4 weeks. The bone marrow was analyzed by flow cytometry. The graph shows the average of absolute numbers of Sca1⁺c-kit⁺lin⁻ stem cells per mouse without and with PTH stimulation in OPN^{+/+} (control) and OPN^{-/-} mice (n=3 or 4).

### DETAILED DESCRIPTION OF THE INVENTION

### Definitions

By “OPN polypeptide” is meant a protein having at least 85% amino acid identity to OPN, or a fragment thereof, that inhibits the survival or proliferation of a hematopoietic stem cell. One exemplary OPN polypeptide is provided at GenBank Accession No. CAA31984.

By “OPN biological activity” is meant negatively regulating the survival or proliferation of a hematopoietic stem cell or stem cell progenitor.

By “OPN nucleic acid molecule” is meant a polynucleotide that encodes an OPN polypeptide or fragment thereof. One exemplary OPN nucleic acid molecule is provided at GenBank Accession No. X13694.

By “OPN inhibitor” is meant a compound that reduces the expression or biological activity of an OPN polypeptide or nucleic acid molecule.

By “allogeneic” is meant cells of the same species.

By “antibody” is meant any immunoglobulin polypeptide, or fragment thereof, having immunogen binding ability.

By “anti-sense” is meant a nucleic acid sequence, regardless of length, that is complementary to the coding strand or mRNA of a nucleic acid sequence. In one embodiment, an antisense RNA is introduced to an individual cell, tissue, organ, or to a whole animals. The anti-sense nucleic acid may contain a modified backbone, for example, phosphorothioate, phosphorodithioate, or other modified backbones known in the art or may contain non-natural internucleoside linkages. Modified nucleic acids and nucleic acid analogs are described, for example, in U.S. Patent Publication No. 20030190659.

By “autologous” is meant cells from the same subject.

By “bone marrow derived cell” is meant any cell type that naturally occurs in bone marrow. Such cells include stromal cells, hematopoietic stem cells, osteoblasts, fibroblasts, adipocytes, endothelial cells, and macrophages.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The dsRNA may contain a modified backbone, for example, phosphorothioate, phosphorodithioate, or other modified backbones known in the art, or may contain non-natural internucleoside linkages.

The term “engraft” as used herein refers to the process of stem cell incorporation into a tissue of interest in vivo through contact with existing cells of the tissue.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids

By “inhibitory nucleic acid” is meant a double-stranded RNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

By “isolated” is meant a material that is free to varying degrees from components which normally accompany it as found in its native state. “Isolate” denotes a degree of separation from original source or surroundings.

By “obtain” is meant purchasing, synthesizing, or otherwise acquiring.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

By “positioned for expression” is meant that the polynucleotide of the invention (e.g., a DNA molecule) is positioned adjacent to a DNA sequence that directs transcription and translation of the sequence.

By “reference” is meant a standard or control condition.

The term “self renewal” as used herein refers to the process by which a stem cell divides to generate one (asymmetric division) or two (symmetric division) daughter cells with development potentials that are indistinguishable from those of the mother cell. Self renewal involves both proliferation and the maintenance of an undifferentiated state.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

The term “stem cell” is meant a multipotent or pluripotent cell having the capacity to self-renew and to differentiate into multiple cell lineages.

By “stem cell generation” is meant any biological process that gives rise to stem cells. Such processes include the differentiation or proliferation of a stem cell progenitor or stem cell self-renewal.

By “stem cell niche” is meant the biological components of a stem cell microenvironment. A stem cell niche includes the OPN-expressing support cells that regulate stem cell survival and generation.

By “stem cell progenitor” is meant a cell that gives rise to stem cells.

By “stromal cell” is meant a cell of the bone marrow present in a hematopoietic stem cell niche.

By “subject” is meant a mammal, including, but not limited to, a human or non-human mammal, such as a bovine, equine, canine, ovine, or feline.

By “support cell” is meant a cell present in a stem cell niche or microenvironment. Support cells include OPN expressing cells that regulate stem cell survival, generation, self-renewal, or differentiation.

By “syngeneic,” as used herein, refers to cells of a different subject that are genetically identical to the cell in comparison.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “xenogeneic,” as used herein, refers to cells of a different species to the cell in comparison.

### Methods of the Invention

The invention generally features therapeutic and prophylactic methods and compositions related to OPN. In one aspect, the invention generally features methods for promoting stem cell survival, self-renewal, or expanding a stem cell population. The invention is based, at least in part, on the discovery that OPN limits stem cell numbers in a hematopoietic stem cell niche. Haematopoietic stem cells derive regulatory information from cells present in the haematopoietic stem cell niche. In one embodiment, methods for modulating stromal cells, which include a variety of cell types, are useful for enhancing haematopoietic stem cell expansion ex vivo or in vivo. The present invention is not limited to methods for enhancing hematopoietic stem cell expansion, but is broadly applicable to a variety of stem cells. Compositions and methods that inhibit OPN expression or activity are useful for expanding stem cell populations in vivo and in vitro. In another aspect, the invention generally features methods for treating a neoplasia. This invention is based, in part, on the discovery that increased levels of OPN induce apoptosis.

### Osteopontin

Osteopontin (OPN), also known as early T cell activation gene-1 (eta-1), is a secreted, highly acidic glycoprotein with pleiotropic effects. OPN binds to cells through arginine-glycine-aspartate (RGD)-mediated interaction with integrins and non-RGD-mediated interactions with CD44 activating multiple different signaling pathways. Stem cells are known to express CD44 and alpha4 integrin, both receptors capable of interacting with OPN. Within bone, OPN is expressed prominently at sites of bone remodeling and cell lined bone surfaces such as the endosteum providing a potential context for stem cells encountering this glycoprotein. Absence of OPN does not affect bone morphology, trabecular spaces associated with stem cell localization or osteoblasts under homeostatic conditions. In addition to being produced by cells of osteoblastic lineage, OPN has been shown to play important roles in chemotaxis, adhesion and proliferation, mediating inflammation and immunity to infectious diseases. For example, granulomatous responses are associated with high levels of OPN expression and OPN can function as a T helper cell-1 (T_{H}1) cytokine, enhancing IL-12 while inhibiting expression of the T_{H}2 cytokine IL-10 (17, 19, 21). Furthermore, OPN can alter sensitivity of hematopoietic cells to other cytokine stimuli. The potential for OPN affecting stem cell function in the niche was high given its abundance in the proper geographic location, receptor expression on stem cells and evidence for it affecting processes in other cells that might be relevant for stem cell physiology. In addition, OPN production is modulated by osteoblast stimulation in vivo resulting in dramatically increased OPN abundance in the areas adjacent to trabecular bone known to serve as the anatomic location of hematopoietic stem cells (3, 4). These data indicated that OPN is produced in a regulated manner, a characteristic that would be expected of a physiologic mediator of niche function.

### Stem Cells

While the specific Examples described below relate to methods of expanding hematopoietic stem cells by reducing OPN expression in a hematopoietic stem cell niche, the invention is not so limited. OPN is likely to function in regulating the size of a variety of stem cell pools. Stem cells of the present invention (e.g., embryonic stem cells, mesenchymal stem-cells, hematopoietic stem cells) include all those known in the art that have been identified in mammalian organs or tissues. The best characterized is the hematopoietic stem cell. The hematopoietic stem cell, isolated from bone marrow, blood, cord blood, fetal liver and yolk sac, is the progenitor cell that generates blood cells or following transplantation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al, U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al, U.S. Pat. No. 5,716,827; Hill, B., et al. 1996.) When transplanted into lethally irradiated animals or humans, hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages observed in vivo.

It is well known in the art that hematopoietic cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

Hematopoietic stem cells can be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated blood products can be enriched for cells having “hematopoietic stem cell” characteristics in a number of ways. For example, the blood product can be depleted from the more differentiated progeny. The more mature, differentiated cells can be selected against, via cell surface molecules they express. Additionally, the blood product can be fractionated selecting for CD34⁺ cells. CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated blood products can be obtained directly from a donor or retrieved from cryopreservative storage.

In preferred embodiments of the invention, the hematopoietic stem cells may be harvested prior to treatment with OPN. “Harvesting” hematopoietic progenitor cells is defined as the dislodging or separation of cells from the matrix. This can be accomplished using a number of methods, such as enzymatic, non-enzymatic, centrifugal, electrical, or size-based methods, or preferably, by flushing the cells using media (e.g. media in which the cells are incubated). The cells can be further collected, separated, and further expanded generating even larger populations of differentiated progeny.

Methods for isolation of hematopoietic stem cells are well-known in the art, and typically involve subsequent purification techniques based on cell surface markers and functional characteristics. The hematopoietic stem and progenitor cells can be isolated from bone marrow, blood, cord blood, fetal liver and yolk sac, and give rise to multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) For example, for isolating hematopoietic stem and progenitor cells from peripheral blood, blood in PBS is loaded into a tube of Ficoll (Ficoll-Paque, Amersham) and centrifuged at 1500 rpm for 25-30 minutes. After centrifuigation the white center ring is collected as containing hematopoietic stem cells.

Stem cells of the present invention also include embryonic stem cells. The embryonic stem (ES) cell has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

Embryonic stem cells are well known in the art. For example, U.S. Pat. Nos. 6,200,806 and 5,843,780 refer to primate, including human, embryonic stem cells. U.S. patent Applications Nos. 20010024825 and 20030008392 describe human embryonic stem cells. U.S. Patent Application No. 20030073234 describes a clonal human embryonic stem cell line. U.S. Pat. No. 6,090,625 and U.S. Patent Application No. 20030166272 describe an undifferentiated cell that is stated to be pluripotent. U.S. Patent Application No. 20020081724 describes what are stated to be embryonic stem cell derived cell cultures.

Stem cells of the present invention also include mesenchymal stem cells. Mesenchymal stem cells, or “MSCs” are well known in the art. MSCs, originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and endothelium. Mesoderm also differentiates to visceral mesoderm, which can give rise to cardiac muscle, smooth muscle, or blood islands consisting of endothelium and hematopoietic progenitor cells. Primitive mesodermal or MSCs, therefore, could provide a source for a number of cell and tissue types. A number of MSCs have been isolated. (See, for example, Caplan, A., et al., U.S. Pat. No. 5,486,359; Young, H., et al., U.S. Pat. No. 5,827,735; Caplan, A., et al., U.S. Pat. No. 5,811,094; Bruder, S., et al., U.S. Pat. No. 5,736,396; Caplan, A., et al., U.S. Pat. No. 5,837,539; Masinovsky, B., U.S. Pat. No. 5,837,670; Pittenger, M., U.S. Pat. No. 5,827,740; Jaiswal, N., et al., (1997). J. Cell Biochem. 64(2):295-312; Cassiede P., et al., (1996). J Bone Miner Res. 9:1264-73; Johnstone, B., et al., (1998) Exp Cell Res. 1:265-72; Yoo, et al., (1998) J. Bon Joint Surg Am. 12:1745-57; Gronthos, S., et al., (1994). Blood 84:4164-73); Pittenger, et al., (1999). Science 284:143-147.

Mesenchymal stem cells are believed to migrate out of the bone marrow, to associate with specific tissues, where they will eventually differentiate into multiple lineages. Enhancing the growth and maintenance of mesenchymal stem cells, in vitro or ex vivo will provide expanded populations that can be used to generate new tissue, including breast, skin, muscle, endothelium, bone, respiratory, urogenital, gastrointestinal connective or fibroblastic tissues.

In certain embodiments, where a stem cell expresses OPN, the stem cell can be treated with an OPN inhibitor. Alternatively, where the stem cell is present in a mixed population of cells that includes a support cell that expresses OPN, such as a stromal cell, the support cell is contacted with an OPN inhibitor or is engineered to express an OPN inhibitor, such as an OPN inhibitory nucleic acid molecule. Biological samples may comprise mixed populations of cells, which can be purified to a degree sufficient to produce a desired effect. Those skilled in the art can readily determine the percentage of stem cells or their progenitors in a population using various well-known methods, such as fluorescence activated cell sorting (FACS). Purity of the stem cells can be determined according to the genetic marker profile within a population. Dosages can be readily adjusted by those skilled in the art (e.g., a decrease in purity may require an increase in dosage).

In several embodiments, it will be desirable to first purify the cells. Stem cells of the invention preferably comprise a population of cells that have about 50-55%, 55-60%, 60-65% and 65-70% purity (e.g., non-stem and/or non-progenitor cells have been removed or are otherwise absent from the population). More preferably the purity is about 70-75%, 75-80%, 80-85%; and most preferably the purity is about 85-90%, 90-95%, and 95-100%. Purified populations of stem cells of the invention can be contacted with an OPN inhibitor before, after or concurrently with purification steps and administered to the subject.

### Stem Cell Culture

Once obtained from a desired source, contacting of a stem cell or support cell present in a stem cell microenvironment with an OPN inhibitor may, if desired, occur in culture. In one embodiment, stem cells are cultured together with support cells that naturally occur in a stem cell microenvironment, or niche. In one embodiment, the support cells are stromal cells or osteoblasts that occur in a hematopoietic stem cell niche. Employing the culture conditions described in greater detail below, it is possible to preserve stem cells of the invention and to stimulate the expansion of stem cell number and/or colony forming unit potential. In all of the in vitro and ex vivo culturing methods according to the invention, except as otherwise provided, the media used is that which is conventional for culturing cells. Appropriate culture media can be a chemically defined serum-free media such as the chemically defined media RPMI, DMEM, Iscove's, etc or so-called “complete media”. Typically, serum-free media are supplemented with human or animal plasma or serum. Such plasma or serum can contain small amounts of hematopoietic growth factors. The media used according to the present invention, however, can depart from that used conventionally in the prior art. Suitable chemically defined serum-free media are described in U.S. Ser. No. 08/464,599 and WO96/39487, and “complete media” are described in U.S. Pat. No. 5,486,359.

Treatment of the stem cells or support cells of the invention with OPN inhibitors may involve variable parameters depending on the particular type of inhibitor used. For example, ex vivo treatment of stem cells or support cells (e.g., bone marrow derived cells or osteoblasts) with RNAi constructs that inhibit OPN expression may have a rapid effect (e.g., within 1-5 hours post transfection) while treatment with a chemical agent may require extended incubation periods (e.g., 24-48 hours). It is also possible to co-culture the stem cells treated according to the invention with additional agents that promote stem cell maintenance and expansion. It is well within the level of ordinary skill in the art for practitioners to vary the parameters accordingly.

The growth agents of particular interest in connection with the present invention are hematopoietic growth factors. By hematopoietic growth factors, it is meant factors that influence the survival or proliferation of hematopoietic stem cells. Growth agents that affect only survival and proliferation, but are not believed to promote differentiation, include the interleukins 3, 6 and 11, stem cell factor and FLT-3 ligand. The foregoing factors are well known to those of ordinary skill in the art and most are commercially available. They can be obtained by purification, by recombinant methodologies or can be derived or synthesized synthetically.

Thus, when cells are cultured without any of the foregoing agents, it is meant herein that the cells are cultured without the addition of such agent except as may be present in serum, ordinary nutritive media or within the blood product isolate, unfractionated or fractionated, which contains the hematopoietic stem and progenitor cells.

### Methods for Creating Genetically Altered Stem Cells

Genetic alteration of a stem cell includes all transient and stable changes of the cellular genetic material which are created by the addition of exogenous genetic material. In one embodiment, a population of cells that includes cells present in a stem cell niche is transfected with an OPN inhibitory nucleic acid molecule (e.g., siRNA, shRNA, antisense oligonucleotides). Such nucleic acid molecules inhibit the expression of OPN. In one approach, an inhibitory nucleic acid molecule is introduced directly into a target cell, such as an osteoblast or other bone marrow derived cell, such that the inhibitory nucleic acid molecule reduces expression of OPN in the cell. In another approach, the target cell is transduced with an expression vector that encodes an inhibitory nucleic acid molecule. Expression of the OPN inhibitory nucleic acid molecule in the target cell reduces OPN expression. Other exemplary genetic alterations include any gene therapy procedure, such as introduction of a functional gene to replace a mutated or nonexpressed gene, introduction of a vector that encodes a dominant negative gene product, introduction of a vector engineered to express a ribozyme and introduction of a gene that encodes a therapeutic gene product. Natural genetic changes such as the spontaneous rearrangement of a T cell receptor gene without the introduction of any agents are not included in this embodiment. Exogenous genetic material includes nucleic acids or oligonucleotides, either natural or synthetic, that are introduced into the stem cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

One method of introducing exogenous genetic material into cells involves transducing the cells in situ on the matrix using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

Because viruses insert efficiently a single copy of the gene encoding the therapeutic agent into the host cell genome, retroviruses permit the exogenous genetic material to be passed on to the progeny of the cell when it divides. In addition, gene promoter sequences in the LTR region have been reported to enhance expression of an inserted coding sequence in a variety of cell types. However, using a retrovirus expression vector may result in (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver an agent and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (UPRT), dihydrofolate reductase (DHFR) (Scharfmann et al., 1991, Proc. Natl. Acad. Sci. USA, 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA, 86:10006-10010), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of an agent in the genetically modified cell. Selection and optimization of these factors for delivery of an is deemed to be within the scope of one of ordinary skill in the art without undue experimentation, taking into account the above-disclosed factors.

In addition to at least one promoter and at least one heterologous nucleic acid, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

### Methods of Using Inhibitory Nucleic Acids

The inhibitory nucleic acid molecules of the present invention may be employed as double-stranded RNAs for RNA interference (RNAi)-mediated knock-down of OPN expression. In one approach, OPN expression is reduced in a stem cell or in a support cell present in a stem cell niche. In one exemplary approach, OPN expression is reduced in a stromal cell or an osteoblast present in a hematopoietic stem cell niche. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and twenty-five consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase m H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

Treatment Methods Related to Stem Cell Expansion

In one aspect, the methods of the invention can be used to treat any disease or disorder in which it is desirable to increase the amount of stem cells and support the maintenance or survival of stem cells. Preferably, the stem cells are hematopoietic stem cells.

Frequently, subjects in need of the inventive treatment methods will be those undergoing or expecting to undergo an immune cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy must be terminated to allow the hematopoietic system to replenish the blood cell supplies before a patient is re-treated with chemotherapy.

Tius, methods of the invention can be used, for example, to treat patients requiring a bone marrow transplant or a hematopoietic stem cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkins lyphoma, or leukaemia.

Disorders treated by methods of the invention can be the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. In addition, methods of the invention can be used to treat damage to the bone marrow caused by unintentional exposure to toxic agents or radiation.

Methods of the invention can further be used as a means to increase the amount of mature cells derived from hematopoietic stem cells (e.g., erythrocytes). For example, disorders or diseases characterized by a lack of blood cells, or a defect in blood cells, can be treated by increasing the pool of hematopoietic stem cells. Such conditions include thrombocytopenia (platelet deficiency), and anemias such as aplastic anemia, sickle cell anemia, fanconi's anemia, and acute lymphocytic anemia. In addition to the above, further conditions which can benefit from treatment using methods of the invention include, but are not limited to, lymphocytopenia, lymphorrhea, lymphostasis, erythrocytopenia, erthrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated—neutropenias, childhood and adult cyclic neutropaenia; post-infective neutropaenia; myelo-dysplastic syndrome; and neutropaenia associated with chemotherapy and radiotherapy.

The disorder to be treated can also be the result of an infection (e.g., viral infection, bacterial infection or fungal infection) causing damage to stem cells.

Immunodeficiencies, such as T and/or B lymphocytes deficiencies, or other immune disorders, such as rheumatoid arthritis and lupus, can also be treated according to the methods of the invention. Such immunodeficiencies may also be the result of an infection (for example infection with HIV leading to AIDS), or exposure to radiation, chemotherapy or toxins.

Also benefiting from treatment according to methods of the invention are individuals who are healthy, but who are at risk of being affected by any of the diseases or disorders described herein (“at-risk” individuals). At-risk individuals include, but are not limited to, individuals who have a greater likelihood than the general population of becoming cytopenic or immune deficient. Individuals at risk for becoming immune deficient include, but are not limited to, individuals at risk for HIV infection due to sexual activity with HIV-infected individuals; intravenous drug users; individuals who may have been exposed to HIV-infected blood, blood products, or other HIV-contaminated body fluids; babies who are being nursed by HIV-infected mothers; individuals who were previously treated for cancer, e.g., by chemotherapy or radiotherapy, and who are being monitored for recurrence of the cancer for which they were previously treated; and individuals who have undergone bone marrow transplantation or any other organ transplantation, or patients anticipated to undergo chemotherapy or radiation therapy or be a donor of stem cells for transplantation.

A reduced level of immune function compared to a normal subject can result from a variety of disorders, diseases infections or conditions, including immunosuppressed conditions due to leukemia, renal failure; autoimmune disorders, including, but not limited to, systemic lupus erythematosus, rheumatoid arthritis, auto-immune thyroiditis, scleroderma, inflammatory bowel disease; various cancers and tumors; viral infections, including, but not limited to, human immunodeficiency virus (HIV); bacterial infections; and parasitic infections.

A reduced level of immune function compared to a normal subject can also result from an immunodeficiency disease or disorder of genetic origin, or due to aging. Examples of these are immunodeficiency diseases associated with aging and those of genetic origin, including, but not limited to, hyperimmunoglobulin M syndrome, CD40 ligand deficiency, IL-2 receptor deficiency, y-chain deficiency, common variable immunodeficiency, Chediak-Higashi syndrome, and Wiskott-Aldrich syndrome.

A reduced level of immune function compared to a normal subject can also result from treatment with specific pharmacological agents, including, but not limited to chemotherapeutic agents to treat cancer; certain immunotherapeutic agents; radiation therapy; immunosuppressive agents used in conjunction with bone marrow transplantation; and immunosuppressive agents used in conjunction with organ transplantation.

Where the stem cells to be provided to a subject in need of such treatment are hematopoietic stem cells, they are most commonly obtained from the bone marrow of the subject or a compatible donor. Bone marrow cells can be easily isolated using methods know in the art. For example, bone marrow stem cells can be isolated by bone marrow aspiration. U.S. Pat. No. 4,481,946, incorporated herein expressly by reference, describes a bone marrow aspiration method and apparatus, wherein efficient recovery of bone marrow from a donor can be achieved by inserting a pair of aspiration needles at the intended site of removal. Through connection with a pair of syringes, the pressure can be regulated to selectively remove bone marrow and sinusoidal blood through one of the aspiration needles, while positively forcing an intravenous solution through the other of the aspiration needles to replace the bone marrow removed from the site. The bone marrow and sinusoidal blood can be drawn into a chamber for mixing with another intravenous solution and thereafter forced into a collection bag. The heterogeneous cell population can be further purified by identification of cell-surface markers to obtain the bone marrow derived germline stem cell compositions for administration into the reproductive organ of interest.

U.S. Pat. No. 4,486,188 describes methods of bone marrow aspiration and an apparatus in which a series of lines are directed from a chamber section to a source of intravenous solution, an aspiration needle, a second source of intravenous solution and a suitable separating or collection source. The chamber section is capable of simultaneously applying negative pressure to the solution lines leading from the intravenous solution sources in order to prime the lines and to purge them of any air. The solution lines are then closed and a positive pressure applied to redirect the intravenous solution into the donor while negative pressure is applied to withdraw the bone marrow material into a chamber for admixture with the intravenous solution, following which a positive pressure is applied to transfer the mixture of the intravenous solution and bone marrow material into the separating or collection source.

It will be apparent to those of ordinary skill in the art that the crude or unfractionated bone marrow can be enriched for cells having desired “stem cell” characteristics. Some of the ways to enrich include, e.g., depleting the bone marrow from the more differentiated progeny. The more mature, differentiated cells can be selected against, via cell surface molecules they express. Enriched bone marrow immunophenotypic subpopulations include but are not limited to populations sorted according to their surface expression of Lin, cKit and Sca-1 (e.g., LK+S+(Lin-cKit⁺Sca1⁺), LK-S+ (Lin-cKit⁺Sca1⁺), and LK+S− (Lin−cKit⁺Sca1⁺)).

Bone marrow can be harvested during the lifetime of the subject. However, harvest prior to illness (e.g., cancer) is desirable, and harvest prior to treatment by cytotoxic means (e.g., radiation or chemotherapy) will improve yield and is therefore also desirable.

Accordingly, the present invention provides methods of treating disease and/or disorders or symptoms thereof which comprise administering a therapeutically effective amount of a pharmaceutical composition comprising a stem cell and or a support cell present in a stem cell niche treated as described herein to a subject (e.g., a mammal, such as a human). Thus, one embodiment is a method of treating a subject having a disease characterized by a lack of blood cells. The method includes the step of administering to the mammal a therapeutic amount of a stem cell, support cell (e.g., stromal cell or osteoblast), or mixture comprising such cell types treated with an OPN inhibitor as described herein sufficient to treat a disease or disorder or symptom thereof, under conditions such that the disease or disorder is treated.

The methods herein include administering to the subject (including a subject identified as in need of such treatment) an effective amount of a stem cell or support cell treated with an OPN inhibitor described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of a pharamaceutical composition comprising a stem cell; support cell (e.g., stromal cell, osteoblast), or mixture of such cell types treated with an OPN inhibitor herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which a lack of blood cells may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with having a reduced number of stem cells, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pretreatment level of Marker can then be compared to the level of Marker in the subject after the treatment commences, to determine the efficacy of the treatment.

### Administration of Stem Cells

Following treatment with a suitable OPN inhibitor, stem cells, support cells, or a mixture comprising such cell types are administered according to methods known in the art. Such compositions may be administered by any conventional route, including injection or by gradual infusion over time. The administration may, depending on the composition being administered, for example, be, pulmonary, intravenous, intraperitoneal, intramuscular, intracavity, subcutaneous, or transdermal. The stem cells are administered in “effective amounts”, or the amounts that either alone or together with further doses produces the desired therapeutic response.

Administered cells of the invention can be autologous (“self”) or non-autologous (“non-self,” e.g., allogeneic, syngeneic or xenogeneic). Generally, administration of the cells can occur within a short period of time following OPN inhibitor treatment (e.g. 1, 2, 5, 10, 24 or 48 hours after treatment) and according to the requirements of each desired treatment regimen. For example, where radiation or chemotherapy is conducted prior to administration, treatment, and transplantation of stem cells of the invention should optimally be provided within about one month of the cessation of therapy. However, transplantation at later points after treatment has ceased can be done with derivable clinical outcomes.

### Stem Cell Related Pharmaceutical Compositions

Following harvest and treatment with a suitable OPN inhibitor, stem cells, support cells (e.g., stromal cells, osteoblasts), or a mixture of cells that include these cells may be combined with pharmaceutical excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells into a subject in need thereof is to incorporate stem cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of stem cells is the quantity of cells necessary to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the particular patient. As few as 100-1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Pharmaceutical Compositions Related to Neoplasia

The invention provides a simple means for identifying compositions (including nucleic acids, peptides, small molecule inhibitors, and mimetics) capable of acting as therapeutics for the treatment of a neoplasia. In particular, the invention provides OPN polypeptide compositions or analogs, or mimetics thereof that are useful for inducing apoptosis in a neoplastic cell. Accordingly, a chemical entity discovered to have medicinal value using the methods described herein is useful as a drug or as information for structural modification of existing compounds, e.g., by rational drug design. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a neoplastic therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the intestinal inflammation or inflammatory bowel disease. Generally, amounts will be in the range of those used for other agents used in the treatment of other diseases associated with neoplasia, although in certain instances lower amounts will be needed because of the increased specificity of the compound. A compound is administered at a dosage that controls the clinical or physiological symptoms of an intestinal inflammation or inflammatory bowel disease as determined by a diagnostic method known to one skilled in the art, or using any that assay that measures the expression or the biological activity of an OPN polypeptide or nucleic acid molecule.

### Screening Assays

Screening methods of the invention can involve the identification of an OPN inhibitor that promotes the expansion of a population of stem cells. Such methods will typically involve contacting a population of cells that include stem cells and cells that express OPN with a suspected inhibitor in culture and quantitating the number of long-term repopulating cells produced as a result. A quantitative in vivo assay (for the determination of the relative frequency of long-term repopulating stem cells) based on competitive repopulation combined with limiting dilution analysis has been previously described in Schneider, T. E., et al. (2003) PNAS 100(20):11412-11417. Similarly, Zhang, J., et al. (2005 Gene Therapy 12:1444-1452) describes the injection of NOD/SCID mice with siRNA-treated lentiviral-transduced human CD34+ cells, followed by the killing of the mice and harvesting of the bone marrow mononuclear cells. The cells were subsequently stained with anti-human leukocyte marker antibodies for FACS analysis allowing the detection of the markers (and, thus, quantitation of the cells of interest). Comparison to an untreated control can be concurrently assessed. Where an increase in the number of long-term repopulating cells is detected relative to the control, the suspected inhibitor is determined to have the desired activity.

In practicing the screening methods of the invention, it may be desirable to employ a cell population that includes not only stem cells, but also support cells. In one embodiment, the support cells express OPN and are treated with a candidate OPN inhibitor prior to or during co-culture with stem cells. In other embodiments, a purified population of stem cells is used. In other methods, the test agent is assayed using a biological sample rather than a purified population of stem cells. The term “biological sample” includes tissues, cells and biological fluids isolated from a subject, as well as tissues, cells and fluids present within a subject. Preferred biological samples include bone marrow and peripheral blood.

Increased amounts of long-term repopulating cells can be detected by an increase in gene expression of certain markers including, but not limited to, Hes-1, Bmi-1, Gfi-1, SLAM genes, CD51, GATA-2, Sc1, P2y14, and CD34. These cells may also be characterized by a decreased or low expression of genes associated with differentiation. The level of expression of genes of interest can be measured in a number of ways, including, but not limited to: measuring the mRNA encoded by the genes; measuring the amount of protein encoded by the genes; or measuring the activity of the protein encoded by the genes.

The level of mRNA corresponding to a gene of interest can be determined both by in situ and by in vitro formats. The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe is sufficient to specifically hybridize under stringent conditions to mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the genes of interest described herein.

The level of mRNA in a sample can be evaluated with nucleic acid amplification, e.g., by reverse transcription-polymerase chain reaction (rtpCR) (Mullis (1987) U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene of interest being analyzed.

In another aspect, screening methods of the invention may be used to identify compositions that induce apoptosis by enhancing the expression or activity of an OPN polypeptide or nucleic acid molecule.

### Test Compounds and Extracts

In general, compounds capable of modulating the expression or activity of an OPN polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. bit. Ed. Engl. 33:2059, 1994; Carell et al., Angew. Chem. Int. Ed Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to decrease the expression or activity of an OPN polypeptide, further fractionation of the positive lead extract is necessary to isolate chemical constituents responsible for the observed effect. Thus, the goal of the extraction, fractionation, and purification process is the careful characterization and identification of a chemical entity within the crude extract that decreases the expression or activity of an OPN polypeptide. Methods of fractionation and purification of such heterogenous extracts are known in the art. If desired, compounds shown to be useful as therapeutics for supporting stem cell expansion are chemically modified according to methods known in the art.

### Kits

The invention provides kits for promoting stem cell survival, growth, or proliferation, as well as kits for enhancing engraftment of a stem cell into a tissue of a subject. In one embodiment, the kit includes a therapeutic composition containing an effective amount of an OPN inhibitor in unit dosage form. In one example, an effective amount of OPN is an amount sufficient to promote stem cell survival or self-renewal in a culture comprising a mixture of cell types that includes stem cells. In other embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an OPN inhibitor is provided together with instructions for administering it to a stem cell culture or to a tissue of a subject. The instructions will generally include information about the use of the composition for the expansion of a stem cell population or for the engraftment of a stem cell population in a tissue. In other embodiments, the instructions include at least one of the following: description of the OPN inhibitor; dosage schedule and administration for the expansion of a stem cell population; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another aspect, the invention provides kits that feature an OPN polypeptide or nucleic acid molecule useful for the treatment of a neoplasia. Such compositions are generally useful for inducing the death of a neoplastic cell (e.g., an aberrant stem cell).

### Neoplastic Therapies

Neoplastic cell growth (e.g., the growth or proliferation of an abnormal stem cell) is not subject to the same regulatory mechanisms that govern the growth or proliferation of normal cells. Compounds that reduce the growth or proliferation of a neoplasm are useful for the treatment of neoplasms. Methods of assaying cell growth and proliferation are known in the art. See, for example, Kittler et al. (Nature. 432 (7020):1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):15814, 2003; Gu et al., Science 302 (5644):445-9, 2003).

Candidate compounds that reduce the survival of a neoplastic cell are also useful as anti-neoplasm therapeutics. In one embodiment, the invention provides for neoplasms that arise from an abnormal stem cell. The neoplasm may be, for example, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, myelodysplastic syndrome, chronic lymphocytic leukemia, polycythemia vera, lymphoma, Hodgkin's disease, Waldenstrom's macroglobulinemia, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, or retinoblastoma. When treating a cancer, it may be desirable to also administer one or more chemotherapeutic agents, biological response modifying agents, and/or chemosensitizers. Desirably, the administration of one or more of these agents is within five days of the administration of the nucleobase oligomer. Exemplary chemotherapeutic agents are adriamycin (doxorubicin), vinorelbine, etoposide, taxol, and cisplatin. While any route of administration that results in an effective amount at the desired site may be used, particularly desirable routes are by intravenous and intratumoral administration.

Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 2742, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate compounds that increase neoplastic cell death (e.g., increase apoptosis) are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Neoplastic cells have a propensity to metastasize, or spread, from their locus of origination to distant points throughout the body. Assays for metastatic potential or invasiveness are known to the skilled artisan. Such assays include in vitro assays for loss of contact inhibition (Kim et al., Proc Natl Acad Sci USA. 101:16251-6, 2004), increased soft agar colony formation in vitro (Zhong et al., Int J. Oncol. 24(6): 1573-9, 2004), the Lewis lung carcinoma (3LL) model of pulmonary metastasis (Datta et al., In Vivo, 16:451-7, 2002) and Matrigel-based cell invasion assays (Hagemann et al. Carcinogenesis. 25: 1543-1549, 2004). In vivo screening methods for cell invasiveness are also known in the art, and include, for example, tumorigenicity screening in athymic nude mice. A commonly used in vitro assay to evaluate metastasis is the Matrigel-Based Cell Invasion Assay (BD Bioscience, Franklin Lakes, N.J.).

If desired, candidate compounds selected using any of the screening methods described herein are tested for their efficacy using animal models of neoplasia. In one embodiment, mice are injected with neoplastic human cells. The mice containing the neoplastic cells are then injected (e.g., intraperitoneally) with vehicle (PBS) or a candidate compound (e.g., an OPN polypeptide or mimetic or an OPN encoding nucleic acid molecule) daily for a period of time to be empirically determined. Mice are then euthanized and the neoplastic tissues are collected and analyzed using methods described herein. Compositions that induce cell death relative to control levels are expected to be efficacious for the treatment of a neoplasm in a subject (e.g., a human patient). In another embodiment, the effect of a candidate compound on tumor load is analyzed in mice injected with a human neoplastic cell. The neoplastic cell is allowed to grow to form a mass. The mice are then treated with a candidate compound or vehicle (PBS) daily for a period of time to be empirically determined. Mice are euthanized and the neoplastic tissue is collected. The mass of the neoplastic tissue in mice treated with the selected candidate compounds is compared to the mass of neoplastic tissue present in corresponding control mice.

### Combination Therapies

Optionally, a stem cell therapeutic may be administered in combination with any other standard therapy for enhancing stem cell survival. Such therapies include the administration of factors that promote stem cell self-renewal, survival, or generation.

OPN nucleic acids or polypeptides may be administered in combination with any other standard neoplasia therapy; such methods are known to the skilled artisan (e.g., Wadler et al., Cancer Res. 50:3473-86, 1990), and include, but are not limited to, chemotherapy, hormone therapy, immunotherapy, radiotherapy, and any other therapeutic method used for the treatment of neoplasia.

The present invention is additionally described by way of the following illustrative, non-limiting Examples that provide a better understanding of the present invention and of its many advantages.

### EXAMPLES

### The Hematopoietic Stem Cell Niche

Components of stem cell niches have generally been defined in terms of cells and signaling pathways. In the murine hematopoietic stem cell niche, the osteoblast is a major niche constituent^{1,2}. Activation of the osteoblast by parathyroid hormone receptor activation increases stem cell numbers and this effect is mediated by Notch1. Deleting BMPR1a similarly increases osteoblasts and causes an increase in stem cells. In both cases, the increase in hematopoietic stem cells is limited to two-fold; an increase that was shown to have physiologic importance, but of surprising uniformity given the varying means of osteoblast activation. To determine whether an osteoblast product could account for this restriction on the stem cell pool size, the osteoblast product, osteopontin (OPN) was analysed.

OPN, also known as early T cell activation gene-1 (eta-1), is a secreted, highly acidic glycoprotein with pleiotropic effects³⁻⁹. OPN binds to cells through arginine-glycine-aspartate (RGD)-mediated interaction with integrins and non-RGD-mediated interactions with CD44 activating multiple different signaling pathways. Stem cells are known to express CD44 and alpha4 integrin, both receptors capable of interacting with OPN^{10,11}. Within bone, OPN is expressed prominently at sites of bone remodeling and cell lined bone surfaces such as the endosteum providing a potential context for stem cells encountering this glycoprotein¹². Absence of OPN does not affect bone morphology, trabecular spaces associated with stem cell localization or osteoblasts under homeostatic conditions¹³. In addition to being produced by cells of osteoblastic lineage, OPN has been shown to play important roles in chemotaxis, adhesion and proliferation, mediating inflammation and immunity to infectious diseases¹⁴⁻¹⁷. For example, granulomatous responses are associated with high levels of OPN expression^{14,15,18} and OPN can function as a T helper cell-1 (T_{H}1) cytokine, enhancing IL-12 while inhibiting expression of the T_{H}2 cytokine IL-10^{15,17,19}. Furthermore, OPN can alter sensitivity of hematopoietic cells to other cytokine stimuli²⁰. The potential for OPN affecting stem cell function in the niche was expected to be high given its abundance in the proper geographic location, receptor expression on stem cells and evidence for it affecting processes in other cells that might be relevant for stem cell physiology.

OPN production is modulated by osteoblast stimulation in vivo resulting in dramatically increased OPN abundance in the areas adjacent to trabecular bone known to serve as the anatomic location of hematopoietic stem cells^{1,2}. These data indicated that OPN is produced in a regulated manner, a characteristic that would be expected of a physiologic mediator of niche function. The role of OPN in the hematopoietic stem cell niche was therefore examined using genetically engineered mice and exogenous OPN. In brief, mice deficient in OPN were found to have an increased stem cell pool size in vivo. Without OPN, there was no significant change in stem cell cycling, but there was increased expression of two ligands known to modify stem cell function, the Notch1 ligand, Jagged1, and the Tie-2 ligand, Angiopoietin-1, accompanied by a decreased rate of stem cell apoptosis. Adding OPN to primitive cells ex vivo increased their apoptotic fraction directly. The ability of OPN to restrict stem cell number was emphasized under conditions of osteoblast stimulation with parathyroid hormone where the expansion of stem cells was increased in the absence of OPN. OPN therefore was discovered to provide a constraining function on stem cell numbers in the hematopoietic stem cell niche and may provide a dampening effect preventing excess stem cell expansion during times of niche stimulation.

### Example 1

### Bone Marrow Osteopontin Production is Altered by Parathyroid Hormone Receptor (PTHr) Activation on Osteoblasts

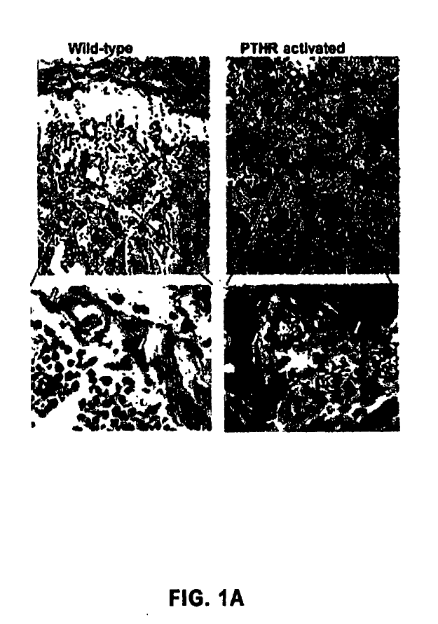

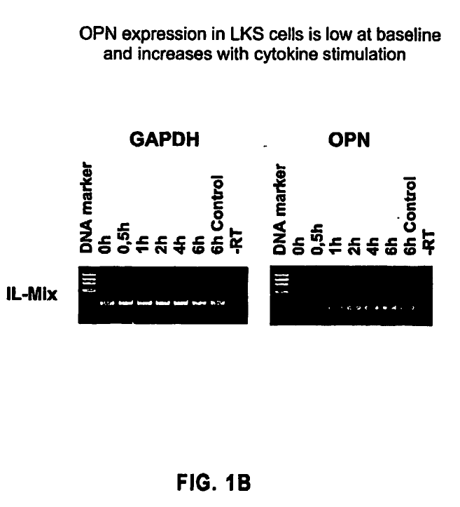

To validate that OPN is produced in a modulated manner at sites relevant for hematopoiesis, immunohistochemistry was performed on tibia sections of wild-type animals or animals having an activated PTHr. In order to focus specifically on osteoblast production of OPN, mice transgenic for a constitutively active PTHr driven by the osteoblast specific collagenα1(I) promoter were used. Production of OPN in the marrow cavity under normal homeostatic conditions was generally in immediate proximity to spindle shaped osteoblasts lining trabecular bone surfaces (FIG. 1A). In contrast, with activated PTHr, OPN staining was markedly increased and extended diffusely from the trabecular surface into the interstitium surrounding hematopoietic cells (FIG. 1A). That osteoblasts were producing this increased OPN was previously demonstrated using combined in situ hybridization and immunohistochemistry²¹. Other hematopoietic cells also expressed OPN in response to cytokine stimuli (FIG. 1B). Taken together, these data indicated that OPN is produced in varying amounts with resulting different distributions affected by cell stimulation. Demonstration of differing production of OPN by the known niche constituent, the osteoblast, suggests a role for OPN in bone marrow homeostasis.

### Example 2

### Expanded Primitive Cell Pool in OPN Deficient Mice

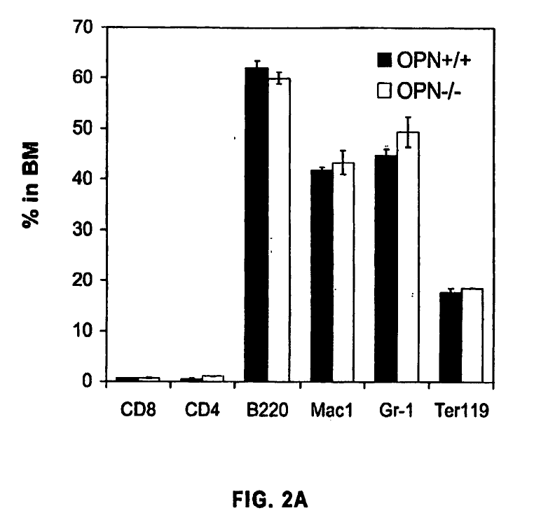

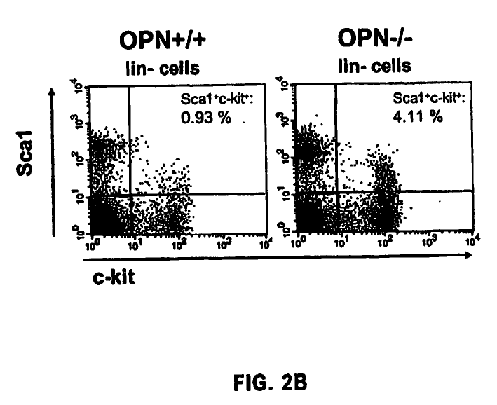

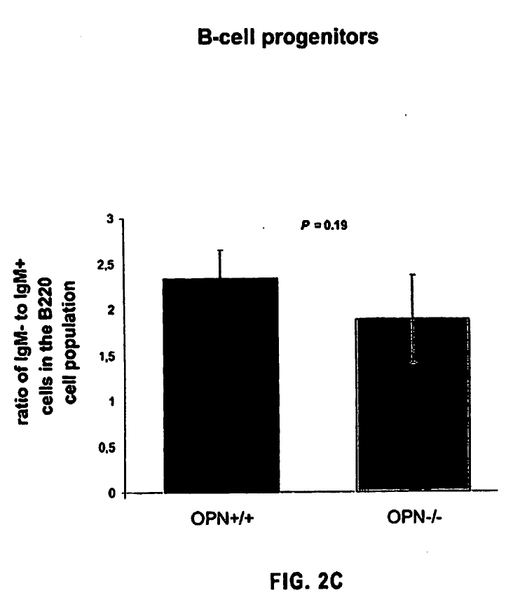

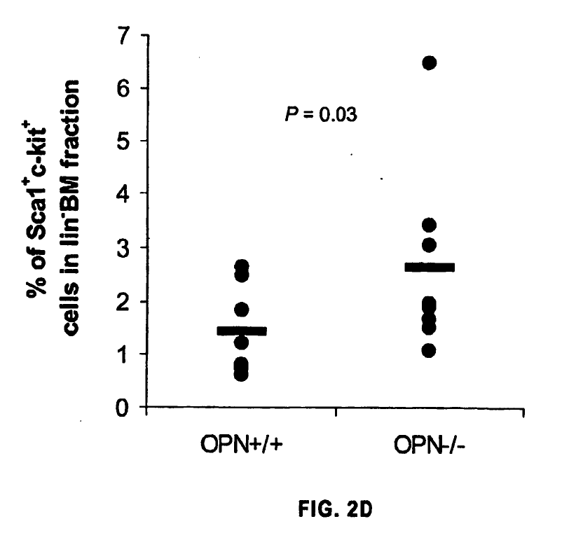

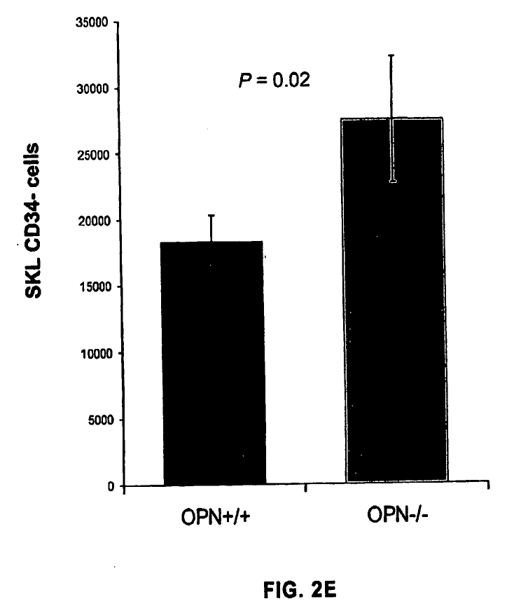

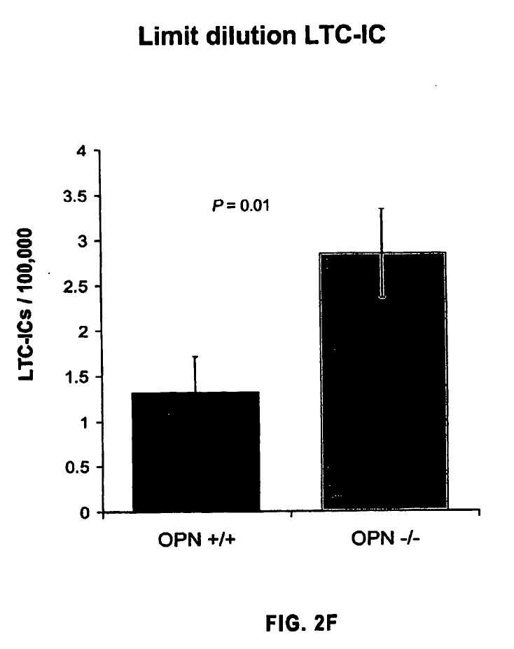

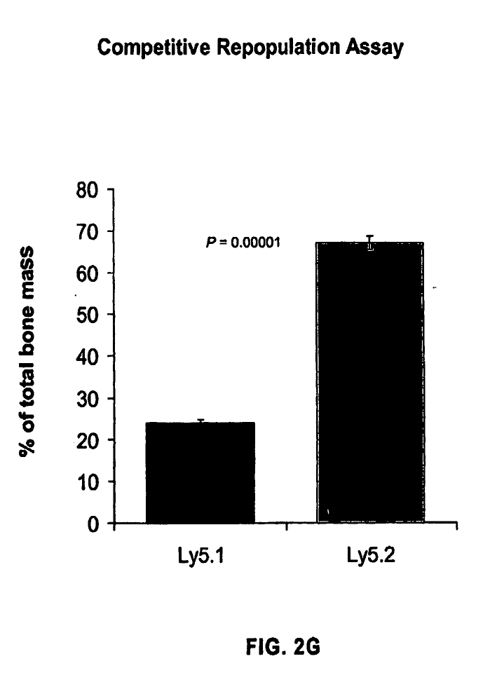

Initially, the bone marrow hematopoietic compartment were characterized under steady-state conditions using animals engineered to be deficient in OPN or their wild-type littermate controls FIG. 2A)¹³. The total cellularity (OPN^{+/+} 54.4±4.7×10⁶ cells vs. OPN^{-/-} 51.4±3.8×10⁶ cells; P=0.31, n=9) and the proportion of differentiated cells, such as B- and T-lymphocytes, granulocytes or erythroid cells, was not altered in the absence of OPN (FIG. 2B). Therefore, OPN-deficiency has a minimal impact on the steady state of more mature blood elements and similarly modest changes in precursor populations as determined by quantitating cells without mature lineage markers (lin⁻)(absolute numbers: OPN^{+/+} 2.6×10^{6±0.2} and OPN^{-/-}3.0×10^{6±0.3} per femur; P=0.16, n=8) or with markers of differentiating erythroblasts (Ter119/CD71) or B cells (B220/IgM- or B220/IgM+) (FIG. 2C). Flow cytometric analysis revealed significantly more primitive cells in the stem cell enriched Sca1⁺c-kit⁺lin⁻ cells in OPN-deficient mice compared with controls (OPN^{+/+} 1.44±0.26% vs. OPN^{-/-} 2.64±0.58% P=0.03, n=8) (Absolute number: 2.92±0.55×10⁴ vs. 4.68±1.12×10⁴ per femur pair, P=0.02, n=8) (FIG. 2D)⁴¹. Within the Sca1⁺c-kit⁺lin⁻ population, the CD34− subset has been defined to further purify cells capable of long-term reconstitution and these cells were also found to be significantly increased in the OPN deficient animals (P=0.02; n=8) (FIG. 2E)⁴².

To assess the impact on cells defined by function, colony assays were initially performed using the methylcellulose colony-forming cell (CFC) assay for progenitors. A significantly lower number of CFCs in the bone marrow of OPN^{-/-} mice was noted (OPN^{+/+} 30.6±4.1 vs. OPN−/−19.05±2.9 colonies per 10⁴ bone marrow cells; P=0.025, n=5). As a measure of more primitive cells, long-term cultures were performed on primary murine bone marrow stroma using a limiting dilution long-term culture-initiating cells (LTC-IC) assay. OPN^{-/-} bone marrow cells gave rise to a significantly higher number of LTC-IC (P=0.01, n=5) FIG. 2F). Of note, on wild-type stroma used in these assays the OPN null cells were able to mature into normal appearing colonies suggesting that OPN deficiency did not intrinsically impair hematopoietic cell differentiation.

To more accurately assess the impact of OPN on the stem cell compartment, cells were admixed in a 1:1 ratio from the wild type and null genotypes and transplanted into lethally irradiated wild type recipients. Twelve weeks after transplantation, the relative abundance of each genotype was quantitated and the OPN^{-/-} cells represented 67.1±1.6% (n=8) of the bone marrow and blood cells (FIG. 2G). The difference between the relative engraftment of OPN^{-/-} to wild type cells was highly statistically significant (P=0.00001) and reflected an approximately 2-fold excess of stem cells present in the OPN−/− donor marrow. Proliferation, apoptosis or other stem cell autonomous effects could all account for these results and were subsequently addressed.

### Example 3

### Transplantation Analysis Demonstrates a Stroma Determined Effect of OPN on Hematopoietic Stem Cells

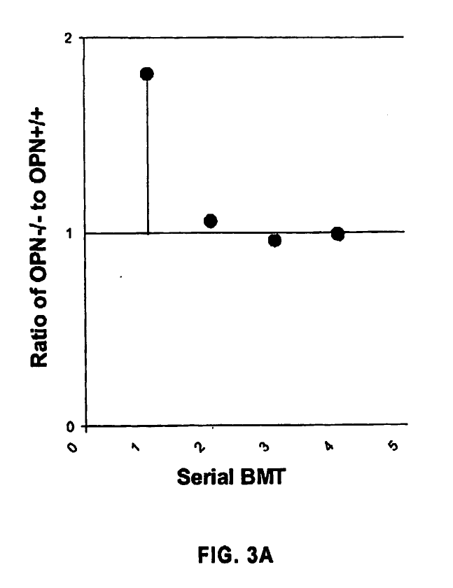

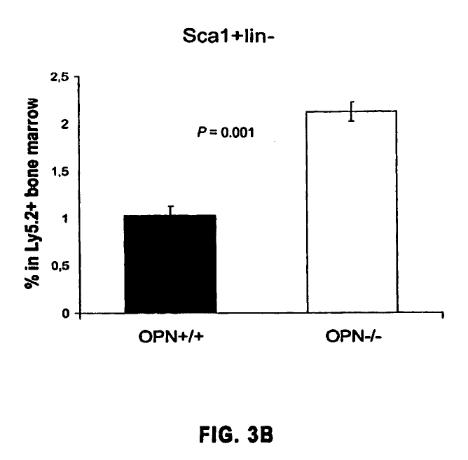

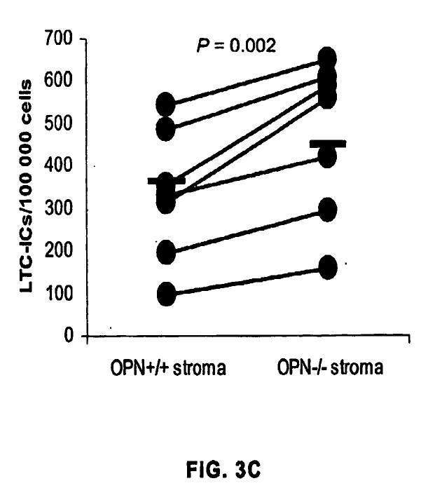

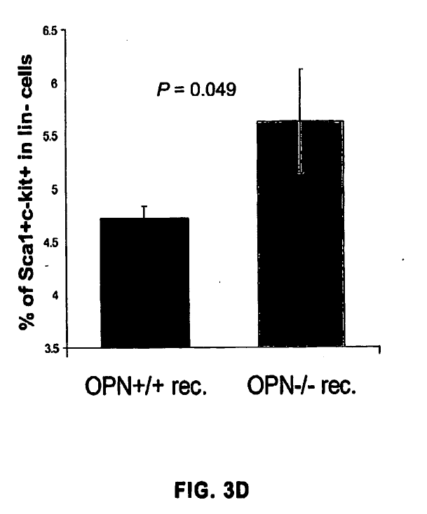

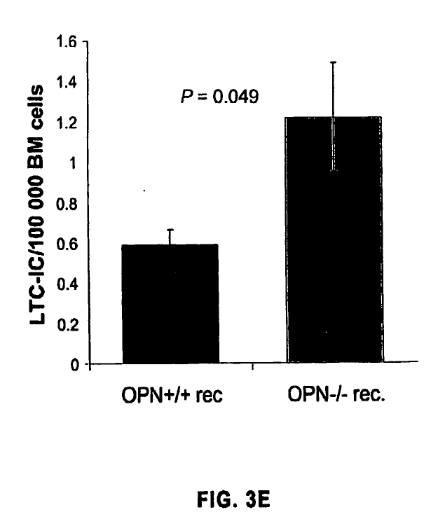

To address whether the impact of OPN was stem cell autonomous or stroma dependent, sequential bone marrow transplantation was carried out based on the reasoning that a stem cell autonomous effect would be retained with each transplant, whereas a non-autonomous or stroma determined effect would not. Bone marrow from OPN^{+/+} or OPN^{-/-} male animals (Ly5.2) was transplanted into lethally irradiated female Ly5.1⁺ mice. Two months after engraftment, 4-8×10⁶ bone marrow cells were used as donor cells and again transplanted in new lethally irradiated Ly5.1⁺ recipients. After a further 3 months, the bone marrows of the secondary recipients were analyzed. There was no difference in the total bone marrow cellularity of animals serially transplanted with OPN^{-/-} or OPN^{+/+} bone marrow cells. Similarly, there was no difference in either proportion or absolute number of the stem cell enriched Sca1⁺kit⁺lin⁻ fraction of Ly5.2⁺ cells in the bone marrow of animals serial transplanted with OPN^{-/-} or OPN^{+/+} cells suggesting unaltered self-renewal ability of OPN deficient stem cells (FIG. 3A). To more accurately quantify the progenitor and primitive cell frequency in the bone marrow of the serially transplanted animals colony forming cell (CFC) and LTC-IC assays were performed. No significant differences between genotypes in either population was detected reflected by these assays. These data document that the alteration in primitive hematopoiesis (increased LTC-IC and decreased CFC) seen in an OPN deficient animal was not persistent when cells from that animal were transplanted into a wild type background. Why the cell numbers would revert back to a level resembling wild type animals has several possible explanations. Without wishing to be bound by theory, it is possible that OPN^{-/-} stem cells do not home as well as OPN^{+/+} cells, hence fewer cells arrive at their supportive niche, accounting for the result.

To directly address the issue of abnormal seeding, in vivo homing assays were performed. Bone marrow cells of OPN^{+/+} or OPN^{-/-} (Ly5.2) mice were transplanted into lethally irradiated wild-type recipients (Ly5.1) (2×10⁷ per animal). Fourteen hours after transplantation the recipient animals were sacrificed and the bone marrow was analyzed by flow cytometry using the surface markers Ly5.1 and Ly5.2 simultaneously with stem cell markers. The proportion of donor cells (Ly5.2) was similar in the bone marrow of animals transplanted with OPN^{+/+} or OPN^{-/-} bone marrow (OPN^{+/+} 3.37±0.4% vs. OPN^{-/-} 2.66±0.2%; P=0.08, n=3). The proportion of Sca1⁺lin⁻ cells, a more primitive subset²², was two-fold higher in the animals transplanted with OPN^{-/-} bone marrow compared with the controls (OPN^{+/+} 1.03±0.1% vs. OPN^{-/-}2.13±0.1%; P=0.001, n=3) (FIG. 3B) reflecting the 2-fold higher proportion of stem cells in the bone marrow of the OPN^{-/-} donor animals prior to transplantation. OPN deficient stem cells therefore do not appear to have any disadvantage in seeding or short term (14 hours) retention in the bone marrow.

To assess the possible role of the microenvironment itself in governing stem cell pool size, stroma from either OPN^{+/+} or OPN^{-/-} mouse bone marrow was cultivated. Sca-1⁺lin⁻ mononuclear bone marrow cells from either genotype were then plated at limiting dilutions in standard LTC-IC conditions. The OPN^{-/-} stroma was capable of supporting LTC-IC greater than wild type stroma (365.5±60.2 LTC-ICs/100 000 cells vs. 450.4±63.1 LTC-ICs/100 000 cells, P=0.002, n=7) (FIG. 3C). These data suggested that stroma was the determinant of primitive pool size and not the primitive cells themselves. This non-autonomous effect on primitive cells supported a role for OPN in the regulatory microenvironment.

To test the in vivo effects of the OPN^{-/-} stroma, wild type cells were transplanted into lethally irradiated OPN^{-/-} or OPN^{+/+} animals. Twelve weeks following engraftment the relative abundance of donor cells was examined by flow cytometry and functional LTC-IC assays. Marrow that had been engrafted in the OPN deficient host demonstrated a statistically significant increase in phenotypic Ly 5.2 Sca1⁺c-kit⁺lin⁻ cells and functional LTC-ICs (4.72±0.11 vs. 5.63±0.49% of Sca1⁺c-kit⁺ cells in the lin⁻ fraction, P=0.049, n=4; 0.59±0.08 vs. 1.22±0.26 LTC-ICs/100 000 cells, P=0.049, n=4) (FIGS. 3D and 3E) closely resembling the OPN null phenotype. Therefore, the microenvironment provided by the OPN deficient animal was able to support a greater number of primitive cells in a stroma dependent manner. These data support the stem cell non-autonomous nature of the OPN^{-/-} effect.

### Example 4

### OPN Deficiency Does Not Affect Cell Cycle Kinetics, but Alters Stromal Jagged1 and Angiopoietin-1 Expression and Primitive Cell Apoptosis

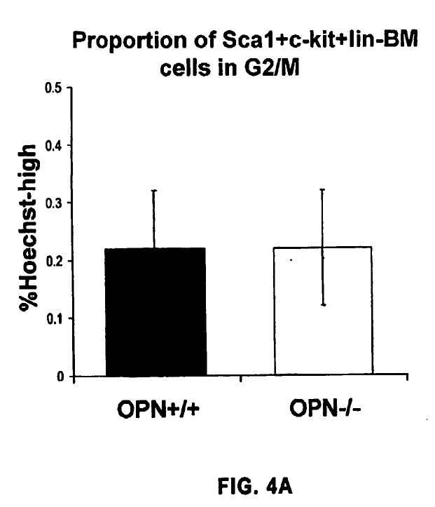

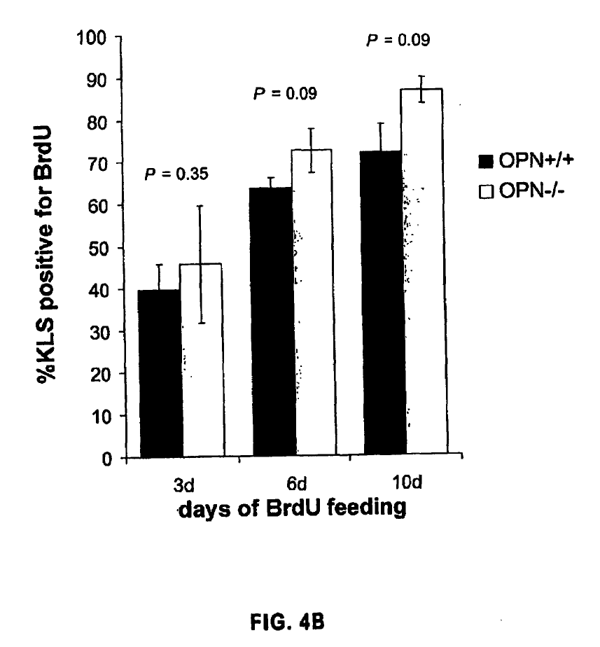

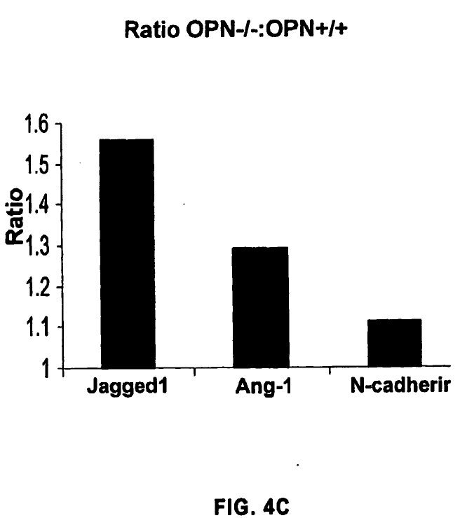

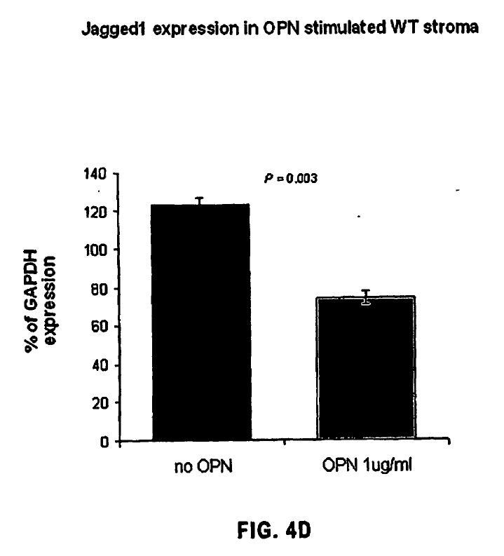

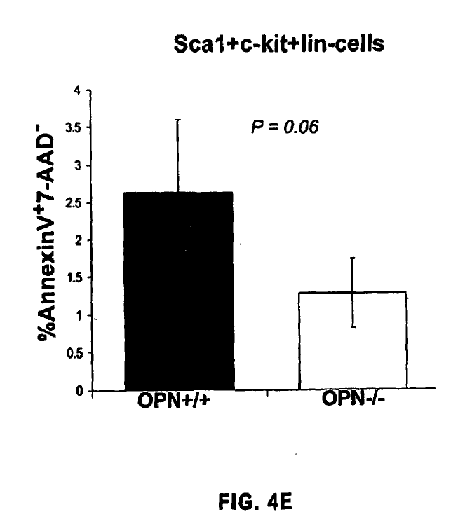

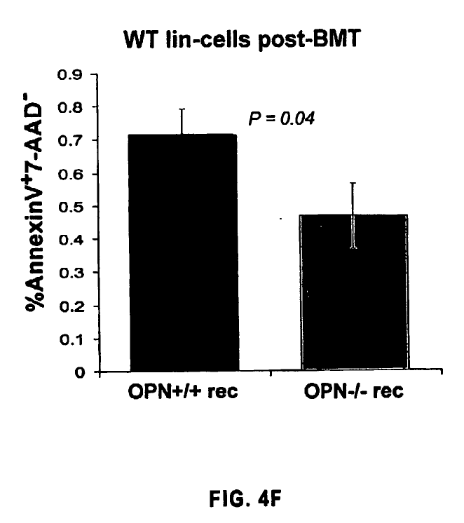

To assess potential mechanisms by which the microenvironment of the OPN deficient animal contributed to the expanded stem cell pool in OPN^{-/-} mice, cell cycle kinetics were assessed. Bone marrow cells were stained with Sca1, c-kit and lineage markers, and the cell cycle status was analyzed by simultaneously staining with the DNA dye Hoechst33342. A similar G0/G1 and S+G2/M percentage of Sca1⁺c-kit⁺lin⁻ was observed in the bone marrow of OPN^{+/+} and OPN^{-/-} animals (S+G2/M OPN^{+/+} 0.22%, OPN^{-/-} 0.22%, pooled bone marrow of 3 animals each) (FIG. 4A). These data indicate an unperturbed cell cycle status of primitive cells in the absence of OPN though it is recognized that they cannot define the interval spent in any phase in a single cycle nor the rapidity of cycling. To better address the latter issue, BrdU labeling was performed, exposing the animals to BrdU in their drinking water for variable intervals and examining the extent of BrdU uptake in primitive subsets of marrow cells by flow cytometry. Modest differences that did not achieve significance were noted between the genotypes at 3, 6 and 10 days (FIG. 4B).

Stem cell expansion may occur without increased proliferation in the context of Notch1 activation where stem cell self renewal is favored over differentiation^{23,24}. Activation of Notch1 on primitive hematopoietic cells in vivo was previously shown to result in an increase in primitive cells, but reduced progenitor cells similar phenotype to that reported herein²³. A link between Notch1 and OPN was reported by Iwata and colleagues who showed that OPN can reduce Notch1 receptor abundance on human CD34+ cells²⁵. Since, the Notch1 ligand, Jagged1 has been shown to be produced by osteoblasts in the hematopoietic stem cell niche and affect stem cell pool size², Jagged1 expression in marrow stromal cells was assessed. An increase in Jagged1 was observed in the OPN deficient animals relative to wild-type controls (P=0.02; n=6) (FIG. 4C). To determine if the reciprocal effect was also true, i.e., that OPN stimulation of wild type cells might decrease Jagged1, marrow stroma was exposed to OPN ex vivo for four hours. Jagged1 was found to be statistically significantly reduced by OPN (FIG. 4D). Other molecular features of the stem cell niche recently defined include N-cadherin¹ and Angiopoietin-1³². The expression of these factors in stroma was examined and non-significant increases in N-cadherin (P=0.08; n=6) and a more pronounced increase in Angiopoietin-1 was observed in the absence of OPN (P=0.02; n=6) (FIG. 4C). Angiopoietin-1 has been defined as a molecule that can increase stem cells, but not be increasing proliferation, rather by enhancing quiescence. These data suggest that the impact of OPN expression is likely multi-factorial, altering features of the niche that in combination change its capacity to nurture primitive hematopoietic cells, but none of which are associated with increased proliferation.

An additional possible mechanism for increasing stem cell numbers without altering cycling kinetics is decreased cell death. To evaluate this, bone marrow cells were stained with stem cell markers, and simultaneously with AnnexinV and the DNA dye 7-AAD to determine the fraction of apoptotic cells indicated by the phenotype AnnexinV⁺7-AAD⁻ (FIG. 4E). A trend toward fewer apoptotic cells in the Sca1⁺c-kit⁺lin⁻ bone marrow stem cell enriched population in OPN^{-/-} mice was detected in comparison with controls (n=4). Additionally, the OPN-deficient bone marrow in serial transplanted animals showed a lower fraction of apoptotic cells in the Sca1⁺c-kit⁺lin⁻ cell population in comparison to controls, suggesting a preserved lower tendency of OPN-deficient stem cells to become apoptotic. Furthermore, when wild type bone marrow was transplanted into either wild type or OPN deficient recipients, lineage negative hematopoietic cells of OPN^{+/+} genotype acquired a decreased apoptosis fraction similar to the OPN deficient animal (FIG. 4F), demonstrating that the basis for the change in apoptosis was stroma dependent. These results suggest that the enlarged stem cell pool in OPN deficient mice may in part be due to enhanced survival, but required further definition.

### Example 5

### Soluble OPN Reduced LTC-IC and Increased the Apoptotic Fraction of Wild Type Cells

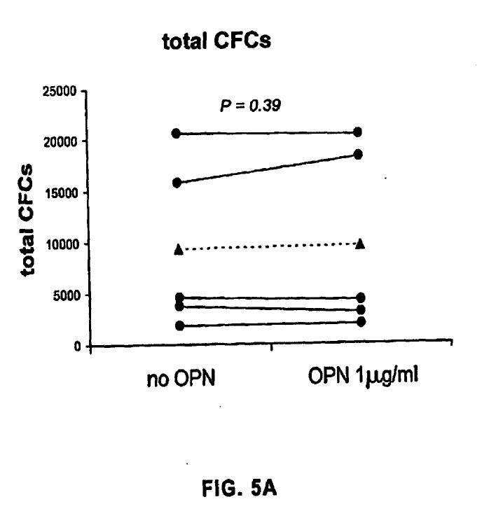

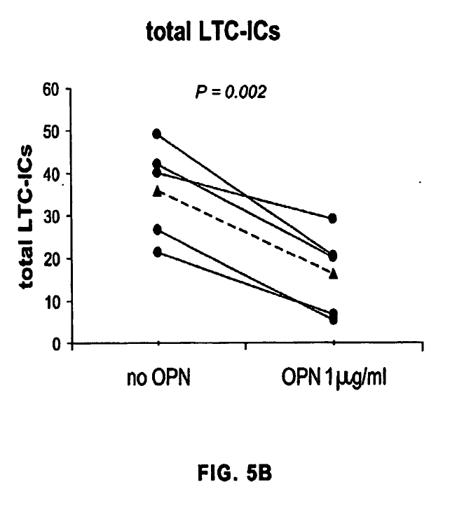

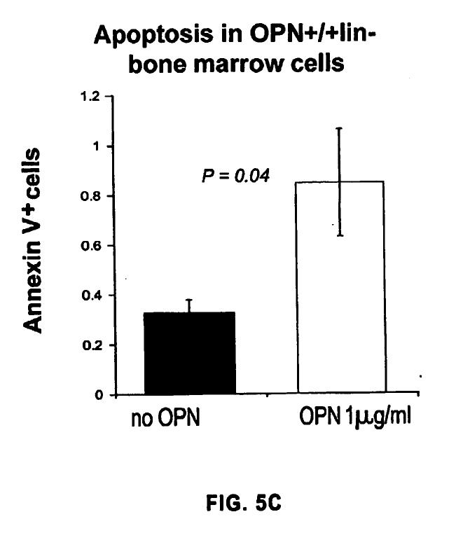

Exogenous OPN was used to assess its potential role in regulating primitive cells directly rather than through the altered expression of other regulators within the niche. Initial experiments cultured Sca1⁺lin⁻ bone marrow cells of C57B1/6 mice in medium containing stem cell factor (SCF), Flt-3, thrombopoietin (TPO), and IL-3 with and without OPN for 7 days. The cells were subsequently counted and analyzed in functional in vitro progenitor and stem cell assays. Addition of soluble OPN led to a lower total cell number with an unperturbed absolute number of CFCs representing hematopoietic progenitor cell activity (n=5) (FIG. 5A). Exogenous OPN led to a significantly lower absolute number of LTC-ICs (without OPN 35.9±5.14 LTC-ICs/well, with OPN 16.41±4.5 LTC-ICs/well; P=0.002, n=5) (FIG. 5 B).

The fraction of apoptotic cells was next analyzed by staining with lineage markers, 7-AAD and AnnexinV. A higher percentage of AnnexinV⁺7-AAD⁻ cells was detected in the lin⁻ cell population cultured with OPN consistent with increased apoptosis (FIG. 5C); a similar effect was seen with Sca⁺lin⁻ cells in the OPN^{-/-} animals and was neutralized with anti-OPN specific antibody. Therefore, the addition of OPN to cell cultures showed the same effect on primitive cell apoptosis that was noted by analysis of the OPN deficient mice in vivo. OPN exerted a pro-apoptotic effect on primitive cells potentially constraining the size of the stem cell pool.

### Example 6

### OPN Restricted Primitive Cell Expansion Induced by Osteoblast Activation

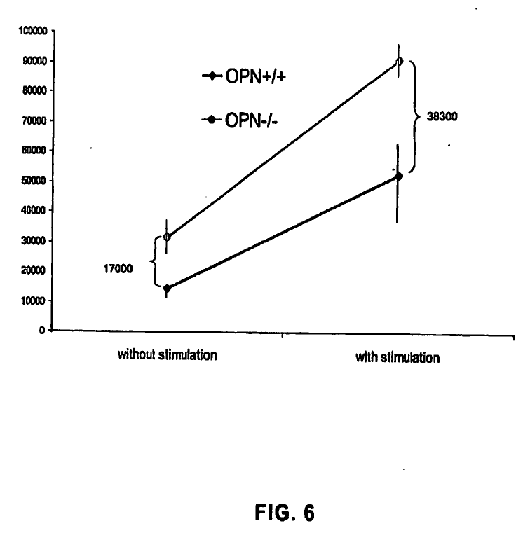

Parathyroid hormone is capable of activating niche osteoblasts and expanding the number of stem cells in vitro and in vivo in a Notch mediated manner. PTH has been shown to be physiologically increased in settings such as myelotoxic ablation with radiation and chemotherapy²⁶. Stimulation with PTH increases OPN production leading to the hypothesis that the degree of stem cell expansion possible by PTH niche activation may be restricted by OPN. Using the OPN null or wild type mouse, the number of primitive cells was assessed following four weeks of PTH stimulation. A difference in the number of Sca1⁺c-kit⁺lin⁻ was noted between the OPN null and wild type mouse prior to PTH (FIG. 6). With PTH treatment, there was an increase in the Sca1⁺c-kit⁺lin⁻ cells in each genotypic background. The magnitude of Sca1⁺c-kit⁺lin⁻ increase induced by PTH was greater in both proportion and absolute number in the null animals (10.0 versus 7.5%, or 5.94×10⁴ vs. 3.82×10⁴ stem cells per femur pair). These data indicate that activation of the niche can increase primitive cells to a greater degree without OPN present in the milieu, arguing that OPN limits the degree of primitive cell increase that can be attained with stimulation of osteoblasts.

The stem cell niche provides a specialized regulatory environment that includes signals to maintain the stem cell pool, protecting it from exhaustion during the life of an organism. Similarly, it provides a context in which stem cells are pushed to differentiate and it likely limits the size of the stem cell pool presumably due to some selective pressure against an excessively abundant stem cell mass. In organisms such as Drosophila, for example, it is well defined that contact of stem cells with hub cells in the germanium are required for preservation of stem cells⁴³. If daughter cells are not in contact with the hub cell, they undergo enforced differentiation resulting in the cessation of cell cycling. In this manner, there is a balance between primitive and differentiated cells and the size of the primitive population does not go beyond the nurturing context of hub cell contact, enforcing a tight control on stem cell number.

This is the first report of a the presence of a similar regulatory relationship in a mammalian system. The results reported herein indicate that OPN expression was modulated by stimulation of the PTH receptor in osteoblasts². OPN production appears to extend the immediate peri-osteoblast area and into stroma away from the endosteal surface. The absence of OPN resulted in an increase in the number of stem cells and the ability to increase primitive cell production when the PTH receptor was activated. Without wishing to be bound by theory, these data are consistent with a model in which OPN restricts the stem cell population and without this mechanism of constraint, expansion exceeds the usual level.

The increase in stem cells when OPN was absent was due to a microenvironmental effect, rather than a stem cell autonomous effect. The effect was not restricted to the bone marrow, as LTC-IC was also noted to be increased in the spleen, an observation that also indicates the change in stem cell pool size was not due simply to redistribution. Localization was one mechanism of OPN action that might have been anticipated given that OPN can engage a number of receptors, including the integrins αᵥ(β₁,β₃ or β₅) and (α₄, α₅, α₈ or α₉)β₁, and is a ligand for certain variant forms of CD44, specifically v6 and/or v7^{8,44-47}. CD44 and integrin α₄ are expressed on primitive hematopoietic progenitor cells and play physiologic roles in stem cell localization^{27,28}. Yet, the effects of OPN noted herein were not associated with altered homing. Nor was there evidence for an altered cycling profile as has been observed in other settings resulting in expanded stem cell numbers such as p21Cip1 or p18INK4c deficiency^{29,30} or HoxB⁴⁸ or Bmi-1 overexpression⁴⁹. Without wishing to be bound by theory, the alteration could be due to a number of influences, including a direct effect of OPN on apoptotic rate. Other factors may contribute to this altered rate, including Jagged1 and Angiopoietin-1. Increased local production of Jagged1, for example, could alter Notch1 activation and affect self-renewal.

It should be noted that there did not appear to be stem cell or hematopoietic cell autonomous changes in self-renewal as evident in the serial transplantation studies, where OPN null stem cells failed to demonstrate persistent increased cell numbers when transplanted into wild-type hosts. Whether there is any link between the findings of decreased apoptosis and up-regulation of Jagged1 or Angiopoietin-1 in the absence of OPN cannot be discerned from these results. Activation of Notch1 in hematopoietic stem cells was previously shown to result in an increased stem cell pool size in vivo with reduced primitive cell production of colonies similar to the phenotype of the OPN null²³. In addition, Notch1 activation was shown to prevent hematopoietic cell death³¹. Angiopoietin-1 has been shown to enhance stem cell interactions with matrix and cell components of the niche^{32,33,34} and to enhance stem cell survival under stress³². Without wishing to be bound by theory, these studies suggest a possible indirect mechanism by which OPN deficiency can change primitive cell populations by altering Jagged1 or Angiopoietin-1 expression; in addition, the data also support a direct functional contribution of OPN. Exogenous OPN provided a pro-apoptotic stimulus in primitive cells that was abrogated with neutralizing antibody to OPN. Therefore, direct and indirect mechanisms likely contribute to the in vivo phenotype of the OPN null.

The results reported herein extend the general concept of matrix proteins regulating neighboring cell functions to that of the stem cell niche. Participation of matrix proteins in creating specialized microenvironments for stem cells that participate in regulating the stem cell pool size adds a novel dimension to the physiologic roles of extracellular matrix constituents. A recent report indicates that the matrix protein, tenascin C, is needed for the proper number and potential of primitive neural cells to be established in the sub-ventricular zone of the central nervous system indicating that extracellular matrix can participate in mammalian stem cell niches³⁵. The results reported herein indicate that a matrix protein whose production is susceptible to modulation, may add a barrier to stem cell expansion upon niche stimulation. Therefore, extracellular matrix components may play a dynamic role in not just establishing the stem cell pool size, but in governing its responsiveness to expansion signals.

These experiments were carried out using the following materials and methods.

### Mice

129/C57BL/6 OPN^{-/-} and 129/C57BL/6 OPN^{+/+} mice are described by Rittling et al. J. Bone Miner. Res. 13: 1101-1111, 1998.

### Cells and Cell Culture.

Mouse bone marrow was obtained from 8-12 week old 129/C57BL/6 OPN^{+/+} and 129/C57BL/6 OPN^{-/-} mice, sacrificed with CO₂. Bone marrow cell and spleen cell suspensions were flushed from femurs and tibias or take from spleen, filtered through 100 μm-mesh nylon cloth (Sefar America Inc., Kansas City, Mo.), and stored on ice until use. Sca1⁺lin⁻ bone marrow wild type cells were obtained from 6-8 weeks old C75B1/6 mice. Bone marrow cells were washed and stained with Sca1⁺ microbeads (Miltenyi Biotec, Bergisch-Gladbach, Germany) and biotinylated lineage antibodies (CD3, CD4, CD8, Gr-1, Mac-1, B220 and Ter119 (Pharmingen, San Diego, Calif.). A positive selection for Sca1⁺ cells followed by a negative selection for Sca⁺lin⁻ cells using streptavidin microbeads was performed in accordance with the manufacurer's instructions (Miltenyi Biotec, Bergisch-Gladbach, Germany). The cells were cultured in IMDM (Gibco-BRL, Rockville, Md.) containing 10% fetal calf serum (FCS), stem cell factor (SCF) [50 ng/ml], Flt-3 [50 ng/ml], thrombopoietin (TPO) [25 ng/ml] and IL-3 [10 ng/ml] (R&D Systems). OPN protein was obtained from R&D Systems.

### Colony Forming Assay.

This assay was used to measure the progenitor cell frequency (CFC) as described by Cheng et al. (Science 287:1804-1808, 2000). Murine stem cell factor (SCF) was used in this study instead of human SCF and cells were plated at only 500 cells/ml.

Long-Term Culture with Limiting Dilutions.

To quantify the stem cells in the bone marrow and spleen cell suspension, the CAFC assay³⁶ was adapted with minor modifications as described in our previous publication³⁷. To measure long term culture-initiating cells (LTC-IC) the semisolid, cytokine containing methylcellulose medium for CFC was overlaid into the wells at week 5 and the colonies were counted at day ten. A limiting dilution analysis software program (Maxrob, kindly provided by Dr. Julian Down, BioTransplant Inc.) was used to calculate the frequency of LTC-ICs in the cell population.

Competitive Repopulation Assay (CRA).

The CRA was used to evaluate the repopulation ability of the OPN^{-/-} bone marrow in irradiated recipient mice^{38,39}. Recipient animals (C57BL/6-Ly5.1, female; Jackson Laboratories) were irradiated with a single dose of 10 Gy 12-16 hours prior to transplantation. The bone marrow donor cells were obtained from 8-10 weeks old, male 129/C57BL/6 OPN^{-/-} and 129/C57BL/6 OPN^{+/+} mice and prepared as above. All leukocytes of these mice are Ly5.2 positive. Congenic competitive bone marrow cells (Ly5.1) were prepared as single cell suspension from male mice. A mixture of equal amounts of cells of the OPN^{-/-} bone marrow cells along with congenic Ly5.1 bone marrow cells were resuspended in Medium 199 and intravenously injected into the lateral tail vein of lethally irradiated Ly5.2 WT or OPN^{-/-} female recipients (n=5 for each group). The mice were sacrificed and bone marrow cells were prepared from those mice and analyzed by flow cytometry.

### Serial Bone Marrow Transplantation

Serial bone marrow transplantation was used to evaluate the ability of stem cells to self-renew. The bone marrow donor cells were obtained from 8-10 weeks old, male 129/C57BL/6 OPN^{-/-} and 129/C57BL/6 OPN^{+/+} mice and transplanted into lethally irradiated wild-type congenic recipients (Ly5.1). The transplanted mice were sacrificed at 2 months and the bone marrow was prepared from those mice. New female recipient mice (n=5 per group) were lethally irradiated and transplanted with 4×10⁶ mononuclear bone marrow cells of the sacrificed animals by injection in lateral tail veins. After 2 months bone marrow cells were harvested from these transplanted mice, analyzed by flow cytometry and again transplanted into lethally irradiated recipients (2^{nd} transplanted mice), which was repeated after further 2 months (3^{rd} transplanted mice) and CFC and LTC-IC assays were performed.

### In Vivo PTH Treatment

6-8 week old wild-type or null male mice were injected with rat PTH (1-34) (Bachem, Torrance, Calif.) (80 μg/Kg of body weight) or vehicle alone intraperitoneally 5×/week for 4 weeks (n=4-6/group). Animals were sacrificed and bone marrow cell isolated and analyzed as above.

### Flow Cytometric Analysis.

Flow cytometry was used to quantify the hematopoietic cells at different stages in the peripheral blood and the bone marrow of the transplanted animals. Bone marrow nucleated cells were labeled with the leukocyte antibodies Ly5.1-PE and Ly5.2-biotin (Pharmingen, San Diego, Calif.), lineage antibodies (CD3-PerCP, CD4-PE, B220-PE, Ter119-PE, (Pharmingen, San Diego, Calif.), CD8-Tri, Gr-1-Tri, Mac1-PE (Caltag)), and stem cell markers (Sca1-Tri and PE, c-kit-Tri (Caltag, Burlingame, Calif.)). To quantify the enriched stem cell phenotype (Sca1⁺lin⁻) in primary animals and in transplanted animals bone marrow cells were stained with biotinylated lineage antibodies (CD3, Ter119 (Pharmingen, San Diego, Calif.), CD4, CD8, B220, IgM, Gr-1 and Mac1 (Caltag, Burlingame, Calif.)), c-kit-APC (Pharmingen, San Diego, Calif.) and Sca1-PE (Caltag, Burlingame, Calif.). The cells were analyzed after labeling with the secondary antibody Streptavidin-PerCP (Becton Dickinson, Franklin Lakes, N.J.). For cell cycle analyses bone marrow cells were incubated with stem cell markers and the DNA dye Hoechst33342. The proportion of apoptotic cells were measured by staining with AnnexinV (Caltag, Burlingame, Calif.) and the DNA-dye 7-AAD (Sigma, St. Louis, Mo.).

### Expression of Jagged1, Angiopoietin 1, N-Cadherin and OPN

The expression of Jagged1, Angiopoietin1 and N-cadherin in bone marrow stroma cells was measured by RT-PCR. Bone marrow stroma cells of OPN+/+ and OPN−/− mice were cultured for 3 to 6 weeks in long-term culture medium and irradiated with 10 Gy to abolish any hematopoietic activity in the culture. After three days cells were lysed with a commercially available phenol and guanidine thiocyanate in a mono-phase solution TRI-reagent (Molecular Research Center (Cincinnati, Ohio) and RT-PCR performed as previously described⁴⁰. The following primers were used: Jagged1: 5′-GTGTGCCTCAAGGAGTATCAG-3′ and 5′-CATAGTAGTGGTCATCACAGG-3′; Angiopoictin1: 5′-GGATTCAACATGGGCAATGTG-3′ and 5′-GGTTCCTATCTCAAGCATGG-3′; N-cadherin 5′-GCAGATFTfCAAGGTGGACG-3′ and 5′-CAGACCTGATTCTGACAAGC-3′; OPN CAAAGTCAGCCGTGAATTCCA-3′ and 5′-AACCCAATAAACTGAGAAAGAAGC-3′. PCR of the reverse transcribed RNA was performed using 25 cycles for Jagged1 and Angiopoietin1 and 27 cycles for N-cadherin. GAPDH transcripts were amplified in 25 PCR cycles. The ethidium bromide-stained gels were photographed and the densitometric results of gene expression were standardized to that of GAPDH expression in the same sample.

### OPN Expression Analysis.

OPN expression in Lin−kit+Sca-1+ (LKS) cells was performed as above following culture of the cells in commercially available culture media, Iscove's modified Dulbecco's medium (IMDM) (Gibco-BRL, Rockville, Md.) containing 10% FCS, SCF [50 ng/ml], Flt-3 [50 ng/ml], TPO [25 ng/ml] and IL-3 [10 ng/ml] (R&D Systems) for the indicated times (FIG. 1B).

### Statistical Analysis.

The significance of the difference between groups in the in vitro and in vivo experiments were evaluated by analysis of variance followed by a one-tailed Student's t-test.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Zhang, et al., (2003) Nature 425:836-841.
2. Calvi, et al., (2003) Nature 425:841-846.
3. Denhardt, et al., (2001) J Clin Invest 107:1055-1061.
4. Denhardt, et al., (1993) Faseb J 7:1475-1482.
5. Rittling, et al., (1999) Exp Nephrol 7:103-113.
6. O'Brien, et al., (1994) Arterioscler Thromb 14:1648-1656.
7. Liaw, et al., (1995) Circ Res 77:665-672.
8. Liaw, et al., (1995) J Clin Invest 95:713-724.
9. Chamnbers, et al., (1996) Lung Cancer 15:311-323.
10. Scott, et al., (2003) Mol Cell Biol 23:9349-9360.
11. Schmits, et al., (1997) Blood 90:2217-2233.
12. McKee, et al., (1993) J Bone Miner Res 8:485-496.
13. Rittling, et al., (1998) J Bone Miner Res 13:1101-1111.
14. O'Regan, et al., (1999) J Immunol 162:1024-1031.
15. Ashkar, et al., (2000) Science 287:860-864.
16. Liaw, et al., (1998) J Clin Invest 101:1468-1478.
17. Chabas, et al., (2001) Science 294:1731-1735.
18. Nau, et al., (1999) Infect Immun 67:4223-4230.
19. O'Regan, et al., (2000) J Leukoc Biol 68:495-502.
20. Lin, et al., (2000) Mol Cell Biol 20:2734-2742.
21. Calvi, et al., (2001) J Clin Invest 107:277-286.
22. Spangrude, et al., (1989) Science 241:58-62.
23. Stier, et al., (2002) Blood 99:2369-2378.
24. Varnum-Finney, et al., (2000) Nat Med 6:1278-1281.
25. Iwata, et al., (2004) Blood 103:4496-4502.
26. Carlson, et al., (1994) Calcif Tissue Int 55:408-411.
27. Dimitroff, et al., (2001) J Cell Biol 153:1277-1286.
28. Antica, et al., (1994) Blood 84:111-117.
29. Cheng, et al., (2000) Science 287:1804-1808.
30. Yuan, et al., (2004) Y Nat Cell Biol 6:436-442.
31. Jang, et al., (2004) J Cell Physiol 199:418-433.
32. Arai, et al., (2004) Cell 118:149-161.
33. Sato, et al., (1998) Int Immunol 10:1217-1227.
34. Takakura, et al., (1998) Immunity 9:677-686.
35. Garcion, et al., (2004) Development 131:3423-3432.
36. Ploemacher, et al., (1991) Blood 78:2527-2533.
37. Cheng, et al., (2000) Nature Medicine 6:1235-1240.
38. Fisher, et al., (1999) Blood 94:1283-1290.
39. Harrison, D. E., (1980) Blood 55:77-81.
40. Stier, et al., (2000) FEBS Lett 467:299-304.
41. Uchida, et al., (1998) J Clin Invest 101:961-966.
42. Osawa, et al., (1996) Science 273:242-245.
43. Xie, et al., (2000) Science 290:328-330.
44. Bayless, et al., (1998) JCell Sci 111 (Pt 9):1165-1174.
45. Denda, et al., (1998) Mol Biol Cell 9:1425-1435.
46. Smith, et al., (1998) Exp Cell Res 242:351-360.
47. Weber, et al., (1996) Science 271:509-512.
48. Sauvageau, et al., (1995) Genes Dev 9:1753-1765.
49. Lessard, et al., (2003) Nature 423:255-260

1. A method of promoting stem cell survival or generation, the method comprising
a) contacting a stem cell or stem cell progenitor and a support cell that expresses osteopontin (OPN) with an OPN inhibitor; and
b) growing the stem cell or stem cell progenitor in the presence of the support cell, to thereby promote stem cell survival or generation.

2. The method of claim 1, wherein the stem cell is selected from the group consisting of a mesenchymal, skin, neural, intestinal, liver, cardiac, prostate, mammary, kidney, pancreatic, retinal and lung stem cell.

3. The method of claim 2, wherein the stem cell is a hematopoietic stem cell.

4. The method of claim 1, wherein the support cell is a cellular component of a stem cell niche.

5. The method of claim 4, wherein the support cell is an osteoblast.

6. The method of claim 1, wherein the generation is by stem cell self-renewal.

7. The method of claim 1, wherein the generation is by proliferation or differentiation of the stem cell progenitor.

8. The method of claim 1, wherein the method reduces apoptosis.

9. The method of claim 1, wherein the method is carried out in vivo.

10. The method of claim 1, wherein the method is carried out in vitro.

11. A method of promoting stem cell survival or generation, the method comprising
a) contacting a stem cell or stem cell progenitor that expresses osteopontin (OPN) with an OPN inhibitor; and
b) growing the stem cell or stem cell progenitor, to thereby promote stem cell survival or generation.

12. The method of claim 1 wherein the stem cell is a hematopoietic stem cell.

13-29. (canceled)

30. A method of increasing the number of self-renewing stem cells in a subject in need thereof, the method comprising the steps of:
contacting an isolated population of cells that comprises at least stem cells and support cells with an OPN inhibitor; and
administering the cells to the subject, thereby increasing the amount of self-renewing stem cells in the subject.

31-48. (canceled)

49. A method for enhancing engraftment of a stem cell into a tissue of a subject, the method comprising
a) contacting a tissue of a subject with an OPN inhibitor; and
b) providing a stem cell to the tissue, thereby enhancing engraftment of the stem cell into the tissue of the subject.

50-54. (canceled)

55. A method of modulating a stem cell niche, the method comprising contacting the niche with an OPN inhibitor, thereby modulating the stem cell niche.

56-62. (canceled)

63. A method for enhancing the hematopoietic stem cell-proliferating activity of a stromal cell comprising contacting the stromal cell with an OPN inhibitor.

64-66. (canceled)

67. The method of claim 49, wherein the stem cell is a hematopoietic stem cell.

68-84. (canceled)

85. A method of inhibiting the survival or proliferation of a neoplastic cell, the method comprising contacting a neoplastic cell with an effective amount of an OPN polypeptide or analog thereof, to thereby inhibit the survival or proliferation of the neoplastic cell.

86. A method of inducing apoptosis in a neoplastic cell, the method comprising contacting a neoplastic cell with an effective amount of an OPN polypeptide or analog thereof, to thereby induce apoptosis in the neoplastic cell.

87-88. (canceled)

89. A method of treating or preventing a neoplasia in a subject in need thereof, the method comprising contacting a cell of the subject with a pharmaceutical composition comprising an effective amount of an OPN polypeptide or analog thereof, to thereby treat or prevent a neoplasia.

90. (canceled)

91. A method of treating or preventing a neoplasia in a subject in need thereof, the method comprising contacting a cell of the subject with a pharmaceutical composition comprising an effective amount of a compound that increases the expression of an OPN polypeptide or nucleic acid molecule, to thereby treat or prevent a neoplasia in the subject.

92-101. (canceled)
